(19) United States
(12) Patent Application Publication
(10) Pub. No.: US 2023/0149477 A1
(43) Pub. Date: May 18, 2023

(54) METHODS OF IMPROVING HEALTH OF YOUNG RUMINANTS

(71) Applicant: The National Institute for Biotechnology in the Negev Ltd., Beer-Sheva (IL)

(72) Inventor: Itzhak MIZRAHI, LeHavim (IL)

(73) Assignee: The National Institute for Biotechnology in the Negev Ltd., Beer-Sheva (IL)

(21) Appl. No.: 17/916,815

(22) PCT Filed: Apr. 7, 2021
(86) PCT No.: PCT/IL2021/050397

§ 371 Date: Oct. 4, 2022

Related U.S. Application Data
(60) Provisional application No. 63/006,116, filed on Apr. 7, 2020.

(51) Int. Cl. A61K 35/74; A23K 10/18; A23K 20/195; A23K 50/10

(52) U.S. Cl. CPC A61K 35/74; A23K 10/18; A23K 20/195; A23K 50/10

(57) ABSTRACT

A method of altering the composition of the microbiome of an adult ruminant is disclosed. The method comprises administering to the ruminant when it is the newborn stage of life, a composition which alters the amount of bacteria of the Akkermansia genus in the microbiome of the newborn ruminant, thereby altering the composition of the microbiome of the adult ruminant.

### RELATED APPLICATIONS

This application claims the benefit of priority of U.S. Provisional Application 63/006,116 filed 7 Apr. 2020, the contents of which are incorporated herein by reference in their entirety.

### SEQUENCE LISTING STATEMENT

The ASCII file, entitled 87074SequenceListing.txt, created on 6 Apr. 2021, comprising 81,157 bytes, submitted concurrently with the filing of this application is incorporated herein by reference.

### FIELD AND BACKGROUND OF THE INVENTION

The present invention, in some embodiments thereof, relates to the use of agents which manipulate microbes in young ruminants and, more particularly, but not exclusively, to agents which manipulate Akkermansia muciniphila.

Ruminants play an important role in meeting the current and growing demand for meat and milk consumed by human. With the world population reaching 9.2 billion by 2050, sustainable ruminant livestock farming has been suggested as a mean to utilize available feed resources within a system to minimize the use of human-edible grains. However, this may conflict with achieving the growing demand, unless proper techniques are developed and implemented to improve the rumen fermentation. It is known that ruminants utilize a wide variety of dietary substrates that are not digestible by the mammals, through microbial fermentation taking place primarily in the rumen. The rumen is the fore-stomach of ruminants, which harbors highly dense and diverse microbial population. Rumen is generally believed to be functioning with solid feed intake and it is physically and functionally different in pre-ruminants compared to that of adult ruminants until the development of the rumen to carry out microbial fermentation.

The rumen microbial fermentation is crucial for the growth and production of ruminants. Thus, the rumen microbial composition and function as well as factors affecting the rumen microbiome (composition and functions), such as diet, age, geographic location, and host species have been well studied in ruminant livestock species. Despite the long history of studies on the rumen microbiota, attempts to manipulate the rumen fermentation are still producing only short-term results. Adult rumen microbiota is resistant to perturbations and original composition is restored following an intervention with exogenous rumen microbiota and diet [Weimer P J. Front microbiol. 2015; 6:296. doi:10.3389/fmicb.2015.00296], suggesting that the microbial manipulation methods are less effective on adult ruminants. Recent studies that focused on early life gut microbiota and its long-term impacts on human health and growth [Francino M P. Pathogens. 2014; 3:769-90. doi:10.3390/pathogens3030769; Subramanian S, et al. Cell. 2015; 161:36-48. doi:10.1016/j.cell.2015.03.013] suggest a potential to manipulate microbiota through early life to obtain beneficial effects during adult life. Indeed, dietary interventions on pre-ruminant rumen microbiota have been successful in achieving fairly persistent and long-term results [Abecia L, et al J Anim Sci. 2013; 91:4832-40. doi: 10.2527/jas.2012-6142; Abecia L, et al. Anim Prod Sci. 2014; 54:1449-54. www(dot)dx(dot)doi(dot)org/10(dot)1071/AN14337; Abecia L, et al. Archaea. 2014; 2014: doi: www(dot)dx(dot)doi(dot)org/10(dot)1155/2014/841463]. However, the knowledge is still limited on the impact of early interventions on adult production.

Additional background art includes WO2019/030752 and US Patent Application No. 2016-0015757.

### SUMMARY OF THE INVENTION

According to an aspect of the present invention there is provided a method of method of altering the composition of the microbiome of an adult ruminant comprising administering to the ruminant when it is at the newborn stage of life, a composition which alters the amount of bacteria of the Akkermansia genus in the microbiome of the newborn ruminant, wherein the composition is:

(i) a microbial composition, wherein at least 5% of the microbes of the composition comprise bacteria of the Akkermansia genus;

(ii) an antibiotic which specifically targets the Akkermansia genus; and/or

(iii) a prebiotic or dietary ingredient which alters the amount of the Akkermansia genus in the newborn, thereby altering the composition of the microbiome of the adult ruminant.

According to an aspect of the present invention there is provided a method of improving a commercially desirable phenotype of an adult ruminant comprising administering to the ruminant when it is at the newborn stage of life, a composition which alters the amount of the genus Akkermansia in the microbiome of the newborn ruminant, wherein the composition is:

(i) a microbial composition, wherein at least 5% of the microbes of the composition belong to the genus Akkermansia;

(ii) an antibiotic which specifically targets the genus Akkermansia; and/or

(iii) a prebiotic or dietary ingredient which alters the amount of Akkermansia in the newborn, thereby improving a commercially desirable phenotype of an adult ruminant.

According to an aspect of the present invention there is provided a method of selecting an agent which improves a commercially desirable phenotype of an adult ruminant comprising:

(a) administering to the ruminant when it is at the newborn stage of life, a composition comprising the agent which alters the amount of the genus Akkermansia in the microbiome of the newborn ruminant; and

(b) analyzing the desirable phenotype in the ruminant at the adult stage of life, when an improvement of the desirable phenotype is indicative that the agent has a positive effect on the desirable phenotype.

According to an aspect of the present invention there is provided a microbial composition comprising a plurality of microbes, wherein at least 10% of the microbes are Akkermansia muciniphila.

According to an aspect of the present invention there is provided a feed comprising the microbial composition described herein.

According to embodiments of the present invention, the agent is selected from the group consisting of:

(i) a microbe;

(ii) an antibiotic which specifically targets the genus Akkermansia; and

(iii) a prebiotic or dietary ingredient which alters the amount of Akkermansia in the newborn.

According to embodiments of the present invention, the newborn stage of life is younger than 15 days.

According to embodiments of the present invention, the bacteria comprises the species Akkermansia muciniphila.

According to embodiments of the present invention, the Akkermansia muciniphila comprises a 16S rRNA gene sequence selected from the group consisting of SEQ ID NOs: 1-179.

According to embodiments of the present invention, the Akkermansia muciniphila comprises a 16S rRNA gene sequence as set forth in SEQ ID NO: 179.

According to embodiments of the present invention, the microbiome comprises the rumen microbiome.

According to embodiments of the present invention, the commercially desirable phenotype is selected from the group consisting of an increase in fertility, a decrease in the propensity to infection, a decrease in methane production, an increase in milk production, an increase in milk quality, an increase in meat quality and an increase in feed efficiency.

According to embodiments of the present invention, the milk quality is selected from the group consisting of a fat content, a lactose content and a protein content.

According to embodiments of the present invention, the infection is selected from the group consisting of brucellosis, campylobacteriosis, cryptosporidiosis, mastitis, Escherichia coli 0157:H7, Q Fever (Coxiella burnetti) infection and Salmonella infection.

According to embodiments of the present invention, the administering is effected more than one time.

According to embodiments of the present invention, the composition is comprised in a feed.

According to embodiments of the present invention, the composition is comprised in a silage.

According to embodiments of the present invention, the composition is comprised in an enema.

According to embodiments of the present invention, the method further comprises administering to the ruminant an antibiotic prior to the administration of the agent. According to embodiments of the present invention, the at least 10% of the microbes of the microbial composition are Akkermansia muciniphila.

According to embodiments of the present invention, the ruminant is a cow.

According to embodiments of the present invention, the ruminant is not weaned.

According to embodiments of the present invention, the microbial composition is formulated as an enema.

According to embodiments of the present invention, the microbial composition further comprises bacteria of the Succinivibrionaceae family, the Lachnospiraceae family and/or the Ruminococcus genus.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B. Age plays an important role in community assembly dynamics. A. The experimental setup consisted of 45 cows (27 delivered vaginally and 18 via C-section), sampled to a minimum of 180 days and over a period of up to 830 days for a third of the cohort (3 years), resulting in high-resolution sampling of more than 1600 samples (bars represents sampling points). The animals were fed with standard dairy-feeding protocols (table on the right), kept under the same conditions, and housed together from the third month of life. B. Principal coordinate analysis (PCoA) based on Bray-Curtis metrics showed clustering of operational taxonomic units (OTUs) according to age and diet (PERMANOVA, P=0.001, two sided test). During microbiome development, age-dependent clustering was identified within the same dietary period, where animals were fed with (i) Diet B and (ii) Diet D (PCoA based on Bray-Curtis metrics, PERMANOVA, P<0.05).

FIGS. 2A-E. Dynamics of the different microbial families is shaped by age and diet. A. Relative abundance of 291 microbial families. All families belonging to the same phylum are colored by different shades of the same color. The main phyla are described in the top left corner of the figure. The Y-axis represents relative abundance and the X-axis represents all of the different samples (n=1634) sorted by sampling day. B. Relative abundance of Bacteroidaceae (blue) and Prevotellaceae (brown). Both belong to the Bacteroidetes phylum. C. Relative abundance of Succinivibrionaceae (blue) from the Proteobacteria phylum and Methanobacteriaceae (brown) from the Archaea domain over time. D. Relative abundance of Verrucomicrobiaceae over time, Akkermansia muciniphila (A. muciniphila; brown) and all other Verrucomicrobia species (blue). E. Relative abundance of Ruminococcaceae (blue) and Lachnospiraceae (brown).

FIGS. 3A-B. The core successional microbiome persists throughout rumen microbiome development, showing age-dependent shifts. A. (i) Heat map showing core successional microbes (n=2544) subjected to hierarchical clustering. Diets are indicated in the color-coded bar above. (ii) Graphical illustration explaining the heat map structure. Each row represents a core OTU and each column represents an animal sampled at a specific time. B. Early-appearing and persistent core successional microbes. Core successional species persistence (Y-axis) as a function of time of appearance (X-axis). Each dot represents the average persistence (number of days) of all microbes that arrived in the ecosystem on the specified day. Species appearance was measured over a 600-day window from first appearance, and persistence was calculated as the mean of the Δ(t_{first appearance}−t_{last appearance}). Purple dots represent core successional microbes, gray dots represent non-core Microbes.

### DESCRIPTION OF SPECIFIC EMBODIMENTS OF THE INVENTION

The present invention, in some embodiments thereof, relates to the use of agents which manipulate microbes in young ruminants and, more particularly, but not exclusively, to agents which manipulate the genus Akkermansia.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The rumen microbial ecosystem and its relationship with the ruminant host is a prime example of obligatory host-microbiome relationships. The host is completely dependent on the microbial community that resides in the upper digestive tract to degrade and ferment the ingested plant biomass, which supports more than two-thirds of its energetic requirements. Moreover the rumen microbiome composition of the adult cow was found to be connected with many of its host attributes and performance.

The present inventors documented the development of the rumen microbiome from birth to adulthood using 16S-rRNA amplicon sequencing data and found that the animals shared a group of core successional species that invaded early on and persisted until adulthood.

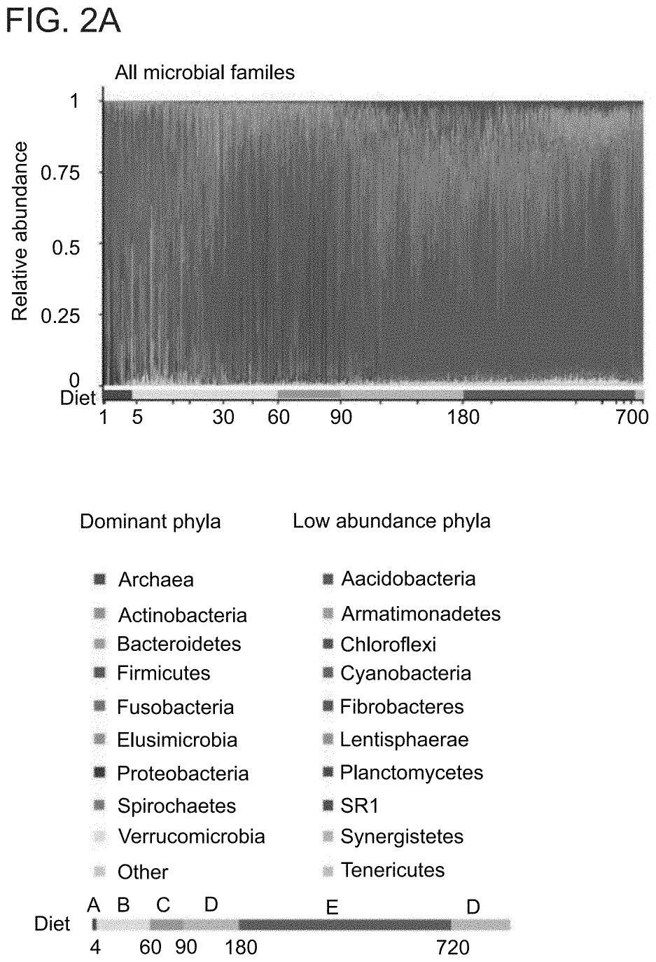

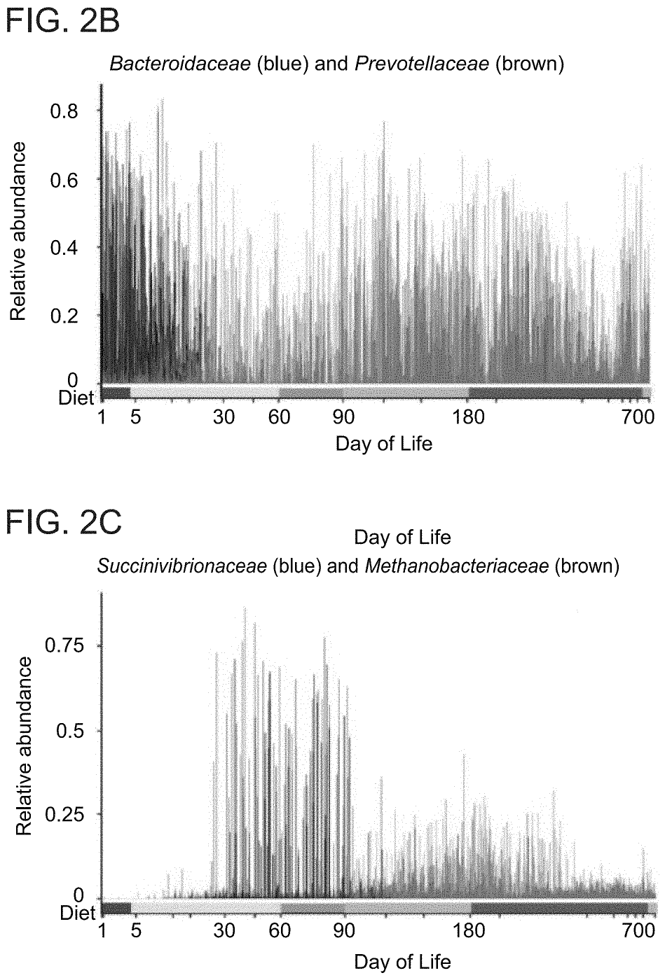

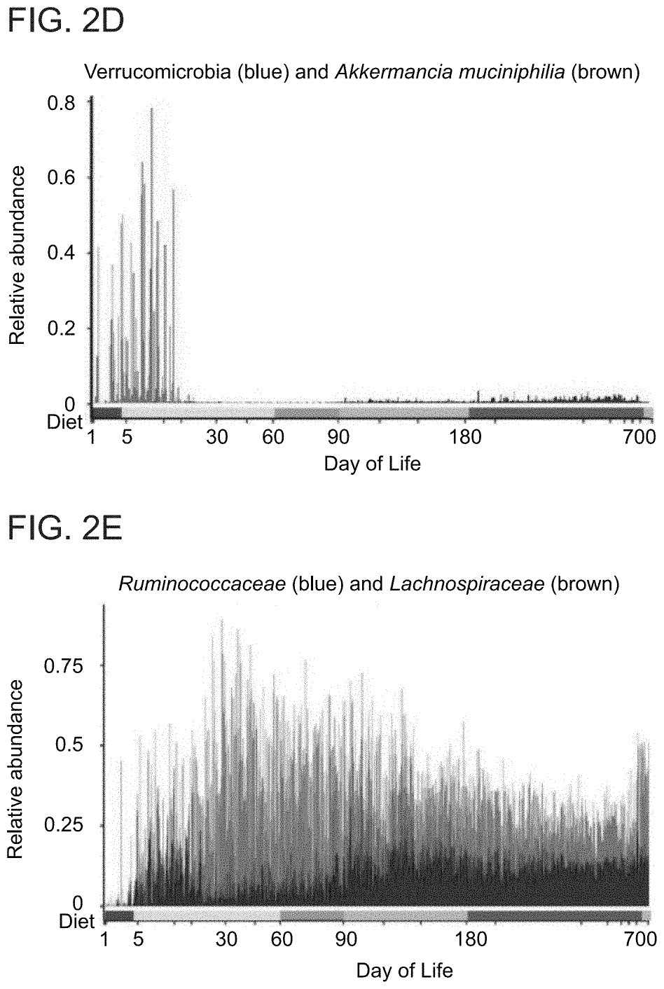

One such species is Akkermansia muciniphila which showed a relative high abundance in the rumen up to the age of one month of the animal, following which the level declined, persisting at this low level of relative abundance until the end of the sampling period (see FIG. 2D). Along with deterministic factors, such as age and diet, the present inventors showed that early arriving species exerted strong priority effects, whereby dynamics of late successional taxa were strongly dependent on microbiome composition at early life stages.

These findings demonstrate the importance of interventions at early life stages to modulate microbiome development, even under the same husbandry regimes.

Thus, according to a first aspect of the present invention there is provided a method of altering the composition of the microbiome of an adult ruminant comprising administering to the ruminant when it is at the newborn stage of life, a composition which alters the amount of bacteria of the Akkermansia genus in the microbiome of the newborn ruminant, wherein said composition is:

(i) a microbial composition, wherein at least 5% of the microbes of the composition comprise bacteria of the Akkermansia genus;

(ii) an antibiotic which specifically targets said Akkermansia genus; and/or

(iii) a prebiotic or dietary ingredient which alters the amount of said Akkermansia genus in said newborn, thereby altering the composition of the microbiome of the adult ruminant.

Akkermansia muciniphila is a Gram-negative, strictly anaerobic, non-motile, non-spore-forming, oval-shaped bacterium. Its type strain is Muc^{T} (=ATCC BAA-835^{T}=CIP 107961^{T}). A. muciniphila is able to use mucin as its sole source of carbon and nitrogen, is culturable under anaerobic conditions on medium containing gastric mucin, and is able to colonize the gastrointestinal tracts of a number of animal species including rumen species.

According to a particular embodiment, the Akkermansia muciniphila comprises a 16S rRNA gene sequence at least 90% identical, 91% identical, 92% identical, 93% identical, 94% identical, 95% identical, 96% identical, 97% identical, 98% identical, 99% identical, to any of the sequences set forth SEQ ID NOs: 1-179.

According to a particular embodiment. the Akkermansia muciniphila comprises a 16S rRNA gene sequence at least 90% identical, 91% identical, 92% identical, 93% identical, 94% identical, 95% identical, 96% identical, 97% identical, 98% identical, 99% identical, 99.9% identical to SEQ ID NO: 179.

Taxonomically, a ruminant is a mammal of the order Artiodactyla that digests plant-based food by initially softening it within the animal's first stomach, known as the rumen, then regurgitating the semi-digested mass, now known as cud, and chewing it again. The process of rechewing the cud to further break down plant matter and stimulate digestion is called “ruminating”. Ruminating mammals include cattle, goats, sheep, giraffes, bison, yaks, water buffalo, deer, camels, alpacas, llamas, wildebeest, antelope, pronghorn, and nilgai.

Ruminating animals contemplated by the present invention include for example cattle (e.g. cows), goats, sheep, giraffes, American Bison, European Bison, yaks, water buffalo, deer, camels, alpacas, llamas, wildebeest, antelope, pronghorn, and nilgai.

The present invention is primarily concerned with methods of treating domesticated ruminants, especially those held for commercial livestock breeding. Thus, in a preferred embodiment of the invention, the ruminant is selected from the group of cattle, goats, sheep and buffaloes.

According to a specific embodiment, the ruminating animal is a cow (e.g. a calf).

The present invention contemplates administering the compositions to newborn ruminants, typically not more than one day old. According to another embodiment, the newborn animals are not more than two days old. According to another embodiment, the newborn animals are not more than three days old. According to another embodiment, the newborn animals are not more than 1 week old. According to another embodiment, the newborn animals are not more than 2 week old. According to another embodiment, the newborn animal is younger than 15 days old. According to another embodiment, the newborn animals are not more than 1 month old. According to a particular embodiment, the newborn ruminant is not weaned.

The present inventors contemplate microbial compositions that increase the amount of bacteria of the Akkermansia genus in the microbiome of the newborn ruminant.

A microbial composition is one which comprises viable microbes (e.g. bacteria).

In one embodiment, at least 50%, 55%, 60%, 65%, 70%, 75%, 80%, 85%, 90%, 95%, 99% of the microbes in the microbial composition are bacteria.

In one embodiment, at least 5%, 10%, 20%, 25%, 30%, 35%, 40%, 45%, 50%, 55%, 60%, 65%, 70%, 75%, 80%, 85%, 90%, 95%, of the bacteria in the compositions are of the genus Akkermansia.

In another embodiment, at least 5%, 10%, 20%, 25%, 30%, 35%, 40%, 45%, 50%, 55%, 60%, 65%, 70%, 75%, 80%, 85%, 90%, 95% of the bacteria of the genus Akkermansia are of the species Akkermansia muciniphila.

The microbial compositions of the present invention may comprise more than 1 species of microbes, 2 species of microbes, 3 species of microbes, 4 species of microbes, 5 species of microbes, 6 species of microbes, 7 species of microbes, 8 species of microbes, 9 species of microbes, 10 species of microbes, 20 species of microbes, 30 species of microbes, 40 species of microbes, 50 species of microbes, 60 species of microbes, 70 species of microbes, 80 species of microbes, 90 species of microbes, 100 species of microbes, 200 species of microbes, 300 species of microbes, 400 species of microbes, more than 500 species of microbes or more than 1000 species of microbes. According to a particular embodiment, the composition comprises between 1-100 species of microbes, 1-50 species of microbes, 1-25 species of microbes, 1-10 species or microbes, 1-5 species of microbes, 10-10,000 species of microbes, between 100-10,000 species of microbes or between 1000-10,000 species of microbes.

The microbial composition may be derived directly from a microbiota sample of a newborn ruminant. In one embodiment, the microbiota sample is a rumen sample. Preferably, the level of Akkermansia muciniphila in the sample is analyzed prior to administration to ensure that there is sufficient amount of Akkermansia muciniphila. Alternatively, the microbial composition may be artificially created by adding known amounts of different microbes. It will be appreciated that the microbial composition which is derived from the microbiota sample of an animal may be manipulated prior to administrating by increasing the amount of a particular species (e.g. increasing the amount of/or depleting the amount of a particular species such as Akkermansia muciniphila). In another embodiment, the microbial compositions are not treated in any way which serves to alter the relative balance between the microbial species and taxa comprised therein. In some embodiments, the microbial composition is expanded ex vivo using known culturing methods prior to administration. In other embodiments, the microbial composition is not expanded ex vivo prior to administration.

Methods of analyzing the level of Akkermansia muciniphila in samples derived from animals are summarized herein below:

In some embodiments, the analyzing comprises determining a level or set of levels of one or more DNA sequences. In some embodiments, one or more DNA sequences comprise any DNA sequence that can be used to differentiate between different microbial types. In certain embodiments, one or more DNA sequences comprise 16S rRNA gene sequences. In certain embodiments, one or more DNA sequences comprise 18S rRNA gene sequences. In some embodiments, 1, 2, 3, 4, 5, 10, 15, 20, 25, 50, 100, 1,000, 5,000 or more sequences are amplified.

Taxonomy assignment of species may be performed using a suitable computer program (e.g. BLAST) against the appropriate reference database (e.g. 16S rRNA reference database).

In determining whether a nucleic acid or protein is substantially homologous or shares a certain percentage of sequence identity with a sequence derived from a known Akkermansia, sequence similarity may be defined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed.

According to one embodiment, in order to classify a microbe as belonging to the Akkermansia genus, it must comprise at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to the genus Akkermansia. According to a particular embodiment, the sequence homology is at least 95%.

In some embodiments, a microbiota sample is directly assayed for a level or set of levels of one or more DNA sequences. In some embodiments, DNA is isolated from a microbiota sample and isolated DNA is assayed for a level or set of levels of one or more DNA sequences. Methods of isolating microbial DNA are well known in the art. Examples include but are not limited to phenol-chloroform extraction and a wide variety of commercially available kits, including QJAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR). In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

According to one embodiment, the microbial composition is not derived from fecal material.

According to another embodiment, the microbial composition is not a rumen sample of the newborn ruminant.

According to still another embodiment, the microbial composition is devoid (or comprises only trace quantities) of fecal material (e.g, fiber).

Prior to administration, the animal may be pretreated with an agent which reduces the number of naturally occurring rumen microbiome (e.g. by antibiotic treatment, examples of which are provided herein below). According to a particular embodiment, the treatment significantly eliminates the naturally occurring rumen microflora by at least 20%, 30% 40%, 50%, 60%, 70%, 80% or even 90%.

The microbes may be administered using a catheter or syringe or may be administered using a tube directly into the rumen.

The microbial compositions described herein may comprise additional bacteria such as those described in WO2019/030752 and US Patent Application No. 2016-0015757, the contents of which are incorporated herein by reference.

In a particular embodiment, the microbial composition further comprises bacteria from the Succinivibrionaceae family, the Lachnospiraceae family and/or the Ruminococcus genus and/or the genus Megasphaera.

In another embodiment, the microbial composition further comprises bacteria Coprococcus catus species and/or the Megasphaera elsdenii species and/or the Clostridium propionicum species and/or Clostridium botulinum species.

The microbes may be administered as a single dose or as a plurality of doses.

In another embodiment, the microbes are administered in the feed of the animal or in the drink of the animal (e.g. as a feed additive).

The ruminants may be fed the feed additive composition of the present invention at the early stage of their life. That is, the ruminant may be fed the feed additive composition of the present invention either by itself or as part of a diet which includes other feedstuffs. The ruminant may be fed the feed additive composition of the present invention continuously, at regular intervals, or intermittently. The ruminant may be fed the feed additive composition of the present invention in an amount such that it accounts for all, a majority, or a minority of the feed in the ruminant's diet for any portion of time in the animal's life. According to one embodiment, the ruminant is fed the feed additive composition of the present invention in an amount such that it accounts for a majority of the feed in the animal's diet for at least the first portion of the animal's lifetime (e.g. 1 week, 1 month).

Examples of additional rumen active feed additives which may be provided together with the feed additive of the present invention include buffers, fermentation solubles, essential oils, surface active agents, monensin sodium, organic acids, and supplementary enzymes.

Also contemplated is encapsulation of the microbes in nanoparticles or microparticles using methods known in the art including those disclosed in EP085805, EP1742728 A1, WO2006100308 A2 and U.S. Pat. No. 8,449,916, the contents of which are incorporated by reference. The microbial compositions may be administered orally, rectally (e.g. as an enema) or any other way which is beneficial to the animal such that the microbes reach the rumen of the animal.

As mentioned, the present inventors also contemplate prebiotic agents which increase the level of Akkermansia.

The term “prebiotic” as used herein, refers to a non-microbial ingredient capable of inducing growth or activity of the genus Akkermansia in the rumen of the animal.

In one embodiment, the composition comprises bacteria which do not compete with Akkermansia for essential resources. In still another embodiment, the composition comprises a metabolite of Akkermansia.

In one embodiment, the composition comprises a bacterial population and a prebiotic.

As mentioned, the present invention also contemplates administration of antibiotic agents which specifically target the Akkermansia genus.

Examples of antibiotics contemplated by the present inventors include, but are not limited to Amikacin; Amoxicillin; Ampicillin; Azithromycin; Azlocillin; Aztreonam; Aztreonam; Carbenicillin; Cefaclor; Cefepime; Cefetamet; Cefinetazole; Cefixime; Cefonicid; Cefoperazone; Cefotaxime; Cefotetan; Cefoxitin; Cefpodoxime; Cefprozil; Cefsulodin; Ceftazidime; Ceftizoxime; Ceftriaxone; Cefuroxime; Cephalexin; Cephalothin; Cethromycin; Chloramphenicol; Cinoxacin; Ciprofloxacin; Clarithromycin; Clindamycin; Cloxacillin; Co-amoxiclavuanate; Dalbavancin; Daptomycin; Dicloxacillin; Doxycycline; Enoxacin; Erythromycin estolate; Erythromycin ethyl succinate; Erythromycin glucoheptonate; Erythromycin lactobionate; Erythromycin stearate; Erythromycin; Fidaxomicin; Fleroxacin; Gentamicin; Imipenem; Kanamycin; Lomefloxacin; Loracarbef; Methicillin; Metronidazole; Mezlocillin; Minocycline; Mupirocin; Nafcillin; Nalidixic acid; Netilmicin; Nitrofurantoin; Norfloxacin; Ofloxacin; Oxacillin; Penicillin G; Piperacillin; Retapamulin; Rifaxamin, Rifampin; Roxithromycin; Streptomycin; Sulfamethoxazole; Teicoplanin; Tetracycline; Ticarcillin; Tigecycline; Tobramycin; Trimethoprim; Vancomycin; combinations of Piperacillin and Tazobactam; and their various salts, acids, bases, and other derivatives. Anti-bacterial antibiotic agents include, but are not limited to, aminoglycosides, carbacephems, carbapenems, cephalosporins, cephamycins, fluoroquinolones, glycopeptides, lincosamides, macrolides, monobactams, penicillins, quinolones, sulfonamides, and tetracyclines.

Antibacterial agents also include antibacterial peptides. Examples include but are not limited to abaecin; andropin; apidaecins; bombinin; brevinins; buforin II; CAP18; cecropins; ceratotoxin; defensins; dermaseptin; dermcidin; drosomycin; esculentins; indolicidin; LL37; magainin; maximum H5; melittin; moricin; prophenin; protegrin; and or tachyplesins.

According to still another embodiment, the agent is capable of downregulating an essential gene of the Akkermansia bacteria.

Thus, for example, the present inventors contemplate the use of meganucleases, such as Zinc finger nucleases (ZFNs), transcription-activator like effector nucleases (TALENs) and CRISPR/Cas system to downregulate the essential gene.

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

By increasing or decreasing the level of Akkermansia bacteria at an early age (as detailed herein above), the present inventors contemplate that the rumen microbiome of the adult will be substantially affected.

As used herein, the term “microbiome” refers to the totality of microbes (bacteria, fungae, protists), their genetic elements (genomes) in a defined environment, e.g. within the rumen of a host. In a particular embodiment, the microbiome refers only to the totality of bacteria in a defined environment, e.g. within the rumen of a host.

The term “adult ruminant” refers to a ruminant who is older than 6 months or in another embodiment, older than 12 months.

The present inventors contemplate that the microbiome of the adult ruminant will be affected for at least one year, at least 2 years, at least 3 years or preferably for the whole life of the ruminant.

Since it is known that changes in the microbiome alters various phenotypes of the adult ruminant, by administering agents which affect the level of Akkermansia in the newborn ruminant, the present inventors contemplate that it is possible to control various phenotypes of the adult ruminant.

In one embodiment, administration of agents which affect the level of Akkermansia in the newborn ruminant, improves the feed efficiency of the ruminant.

As used herein, the term “feed efficiency” refers to the ability of the animal to extract energy from its food. The feed efficiency is the difference between an animal's actual feed intake and its predicted feed intake based on its production level and body weight. Thus, an animal with “a high” feed efficiency is one that produces more milk or weighs more that what is predicted based on its feed intake. An animal with “a negative” feed efficiency is one that produces less milk or weighs less than what is predicted based on its feed intake. In one embodiment, the energy efficiency is measured using the residual feed intake (RFI) method (Koch et al., 1963) and may be calculated according to national Research Council 2001 formulas. The expected RFI values for each cow may be calculated based on a multiple regression equation.

In another embodiment, administration of agents which affect the level of Akkermansia in the newborn ruminant, reduces the methane production of the ruminant.

The term “methane production” refers to an amount of methane emitted by the animals per se or produced by the microbiome. It may be measured in units of g per day or g per kg of dry matter intake.

Other exemplary phenotypes that may be affected by administration of agents which affect the level of Akkermansia in a ruminating animal is a propensity (i.e. likelihood) to a disease. The present invention contemplates that by providing the compositions described herein, it may be possible to avoid or delay the development of a disease or condition and/or lessen the associated symptoms. According to one example, the disease is an infectious disease.

Non-limiting examples of infections for which it may be desirable to lower predisposition to include any one of brucellosis, campylobacteriosis, cryptosporidiosis, mastitis, Escherichia coli 0157:H7, Q Fever (Coxiella burnetti) infection and Salmonella infection.

Another exemplary phenotype that the present invention contemplates that may be affected by administration of agents which affect the level of Akkermansia in a ruminating animal is fertility. Thus, for a male animal, it may be desirable to increase virility. Correspondingly, for a female animal, it may be desirable to increase her ability to be impregnated.

Another exemplary phenotype that the present invention contemplates may be affected by administration of agents which affect the level of Akkermansia in a ruminating animal is milk production. The phenotype may refer to milk quantity or milk quality (e.g. fat content, lactate content, protein content etc.).

Still another exemplary phenotype that the present invention contemplates that may be affected by administration of agents which affect the level of Akkermansia in a ruminating animal is quality of meat production. Exemplary phenotypes include muscle:fat ratio.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Animal Handling and Sample Collection

Calves (Holstein-Friesian breed) were divided into two groups: those born by C-section (n=18) and those born by vaginal delivery (n=27). Caesarean section is a routine practice for specific breeds of cows⁶³, nevertheless, it is not a common practice among most dairy cow breeds. However, in the context of this study, this procedure was used as a disturbance of the colonizing communities created early in life, which enabled us to ask whether it could affect microbiome assembly dynamics throughout the experimental period.

Animals were subjected to conventional housing and growth practices in experimental facility. Briefly, calves were immediately separated from their mothers after calving to prevent vertical transmission of microbes from the mother's oral microbiome to their infants. Calves were housed in individual kennels for 90 days. Thereafter, all animals were housed together in corrals. After 90 days, they were transferred to cohabitation groups. These groups were separated according to their diet, age and sex. Animals were consecutively transferred from one dietary/age group to the next, in order to keep the different groups as homogeneous as possible. The overall cohort, 19 males (Males were not castrated) and 26 females, was divided into two groups, C-section-delivered calves (n=18) and vaginally delivered calves (n=27). Males were removed from the herd between the ages of 6 to 8 months. Females were sampled for a period spanning 8 to 28 months. Females underwent artificial insemination around the age of 480 days and gave birth at around 725 days. Overall, 1634 samples were collected, 1062 samples from vaginally delivered cows and 573 samples from C-section delivered cows.

Dietary Regime

Calves were fed solely colostrum for the first 3 days after calving. From day 4 until 2 months of age (60 days), calves were fed milk replacer and starter mixture ad libitum. After 60 days, calves were weaned and fed only starter mixture until 90 days of age. From day 90 to 180, calves received a low-fiber diet. From day 180 to ˜725 days, animals were fed a high-fiber diet. After calving, a low-fiber diet similar to that supplied between 90 and 180 days was provided.

Sampling Regime

Rumen sampling was carried out using a custom-made stomach tube (Metal Systems, Kiryat Gat, Israel), which was specifically designed for this study with a length of 2500 mm and diameter of 12 mm. This stomach tube was used throughout the entire experiment for all animals. The design of diameter and length of the tube was based on the physiology of pre-ruminant animals and aimed to reach the ventral part of the rumen from birth to adulthood. The manufacturing of the tube included electropolishing, which minimized injuring the esophagus. The sampling protocol was similar to that of Jami et al (2013) and Shabat et al (2016)^{14,18}, where in younger calves the inserted length of the tube for rumen sampling was based on initial calibration experiments. In each sampling, the stomach tube was connected to a vacuum pump only when it reached the ventral part of the rumen.

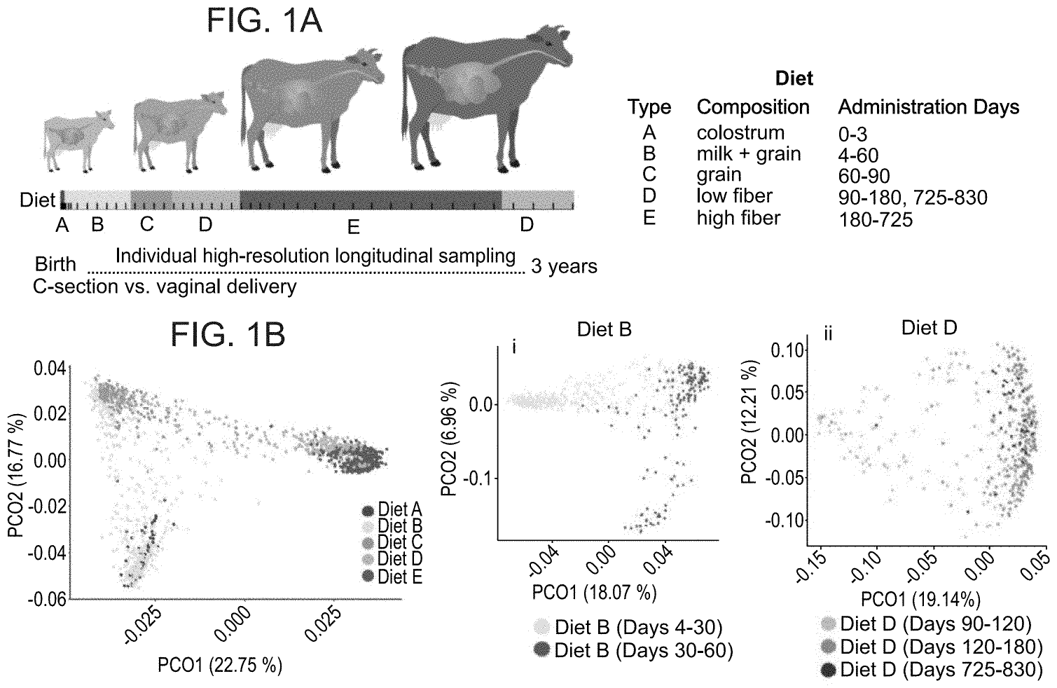

After birth, rumen fluid samples were taken from the newborn calves daily, from day 0 to day 7, due to a previous study in our laboratory that revealed a rapid dynamic changes in microbial composition immediately after birth¹⁸. Rumen content was sampled twice more between day 7 and day 15. After that, rumen content was collected weekly until weaning. Upon weaning on day 60, rumen fluid was collected weekly until at least 220 days of age, after which samples were collected once a month. Experimental setup and dietary regimes are shown in FIG. 1A.

Delivery by C-Section

C-section was performed according to the protocol approved by the Animal Policy and Welfare Committee of the ARO. Anesthesia was administered to the mothers paravertebrally using lidocaine and adrenaline. The mother was shaved locally, scrubbed with povidone-iodine solution and washed with isopropanol; the C-section was performed using the left-flank laparotomy approach. The mother was then given penicillin and aminoglycosidic antibiotic and recovery was followed until involution of the uterus.

Bacterial Extraction

Thawed rumen samples were transferred to centrifuge bottles and kept on ice for no more than 20 min before processing. Rumen samples were processed as described previously⁶⁴. The samples were centrifuged at 10,000 g and the pellet was dissolved in extraction buffer [100 mM Tris-HCl, 10 mM ethylenediaminetetraacetic acid (EDTA), 3% w/v Tween 80, 0.15 M NaCl, pH 8.0]; 1 g of pellet was dissolved in 4 ml of buffer and incubated at 4° C. for 1 h, as chilling has been shown to maximize the release of particle-associated bacteria from ruminal contents⁶⁵. The suspension was then centrifuged at 500 g for 15 min at 4° C. to remove ruptured plant particles while keeping the bacterial cells in suspension. The supernatant was then passed through four layers of cheesecloth, centrifuged (10,000 g, 25 min, 4° C.) and the pellets were kept at −20° C. until DNA extraction.

DNA Extraction

DNA extraction was performed as previously described⁶⁴. Briefly, cells were lysed by bead disruption with phenol followed by phenol/chloroform DNA extraction. The final supernatant was precipitated with 0.6 volume of isopropanol and resuspended overnight in 50-100 μl TE (10 mM Tris-HCl, 1 mM EDTA), then stored at −20° C.

Animal Genotyping

Genomic DNA extracts from 36 animals were loaded into a bovine SNP 50K chip, which is targeted at 54,609 common SNPs that are evenly spaced along the bovine genome (Illumina). The SNP chip model used was Illumina bovine SNP50-24 v3.0, catalog no. 20000766, and it was processed according to the manufacturer's protocol at the Genomics Center of the Biomedical Core Facility, Technion, Israel.

Genotype Data Quality Control

QC was performed with the PLINK⁶⁶ program, with the following parameters: -cow-file isgenotype_all-maf 0.05-geno 0.05-mind 0.05-recode12. SNPs that were not genotyped in more than 5% of the individuals were removed. Similarly, individuals were removed from the analysis if they had been genotyped in less than 95% of the loci (SNPs) covered by the SNP chip. Three individuals were removed because of low genotyping, 3,001 SNPs were removed because of “missingness” in the genotyped populations, and 15104 SNPs failed the minor allele frequency (MAF) criteria. The total number of SNPs passing QC was 38359.

Estimating Kinship Matrix

Cows kinship matrix was built based on autosomal QC-filtered SNP values similarity between cows, by IBS approach using EMMAX⁶⁷ with command line parameters: emmax-kin-intel64 -v -s -d 10.

16S rRNA Gene Amplicon Sequencing

Sequencing protocols are identical to the earth microbiome protocols. Amplification of 16S rRNA gene from the ruminal samples was performed according to Caporaso et al.⁶⁸ for the V4 region, using the primers 515F (5′-GTGCCAGCMGCCGCGGTAA-3′—SEQ ID NO: 180) and 806R (each reverse primer contained a different 12-bp index). The protocol was performed under the following conditions: 94° C. for 15 min, followed by 35 cycles of 94° C. for 45 s, 50° C. for 60 s and 72° C. for 90 s, and a final elongation step at 72° C. for 10 min. The PCR product (380 bp) was cleaned using the DNA Clean & Concentrator™ kit (Zymo Research) and quantified for fragments containing the Illumina adaptors. Sequencing was performed using the Illumina Miseq sequencer. For controls in all our runs, we used non-template controls for each of the samples, and therefore all samples were monitored for contamination. The product was quantified using a standard curve with serial DNA concentrations (0.1-10 nM). Finally, the samples were diluted to a concentration of 4 nM and prepared for sequencing according to the manufacturer's instructions. The normalized samples were then unified and sequenced by the paired-end method.

Quality Control

Data quality control and analyses were mostly performed using the QIIME 1.9 pipeline⁶⁸. First, after paired ends were joined, reads were demultiplexed, and read-quality filtering was performed using the default settings of the “split_libraries_fastq” command. Total read count for all 1634 samples was 55,493,183 reads, with an average of 33,270±24,875 reads per sample. All samples were then subsampled to 10,000 reads per sample. The next step was to align the obtained sequences to define OTUs for eventual taxonomy assignment. The Uclust method was used to cluster the reads into OTUs using the pick_otus command at 97% similarity. Taxonomy was assigned using the Ribosomal Database Project (RDP) classifier against the 16S Greengenes reference database (blog(dot)qiime(dot)org), designated as ‘most recent Greengenes OTUs’. After an OTU table was created, singletons and doubletons were discarded.

Similarity Measurement

For similarity measurement between the bacterial communities in the samples, the Bray-Curtis and UniFrac distance similarity indices were used to compare samples according to both presence and absence of OTUs and relative abundance of OTUs between samples. A PCoA eigenvalues table was calculated using the Bray-Curtis similarity matrix. The beta_diversity.py and principal_coordinates.py Qiime scripts were used to calculate beta-diversity indices. Separation of the different samples within diet clusters was performed using PERMANOVA (qiime script: compare_categories.py). Random Forest classifier was applied using qiime supervised learning.py command.

Core Successional Microbes Analysis

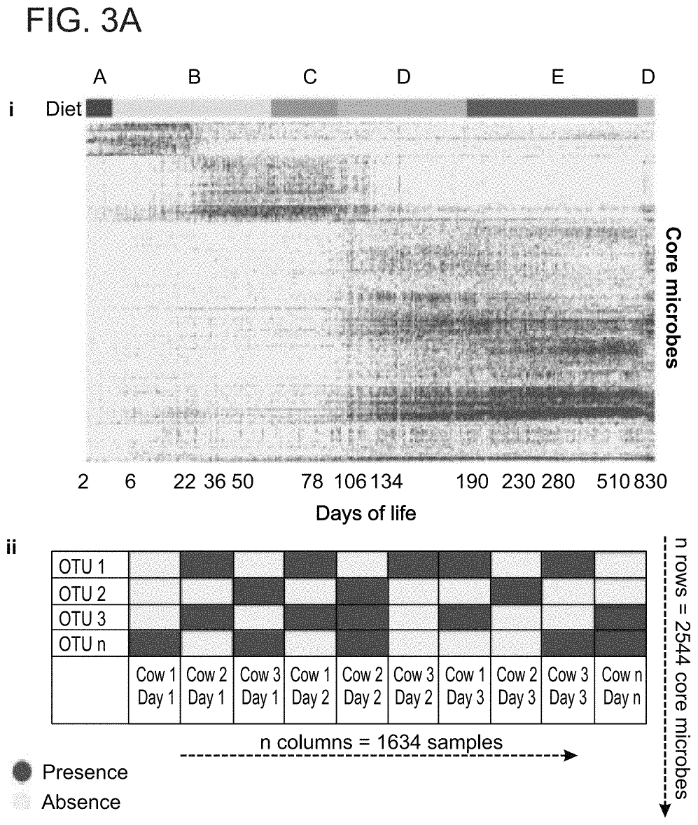

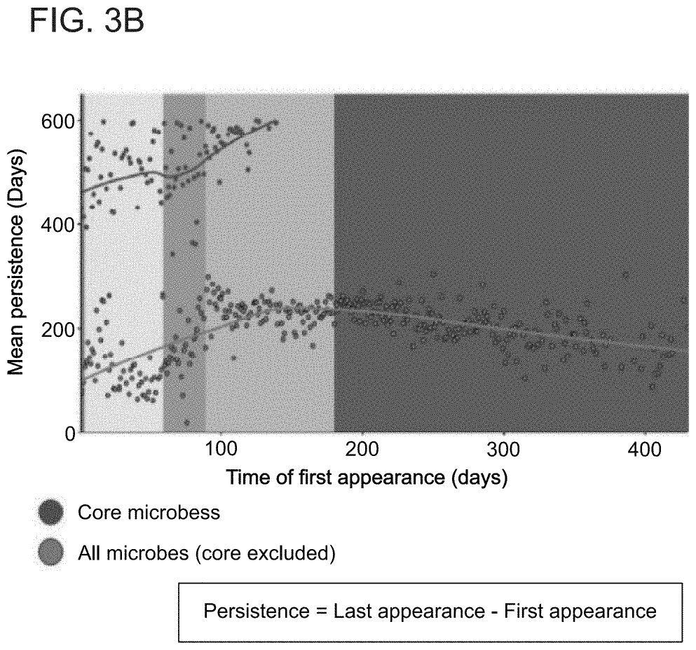

Core successional microbes were calculated using compute_coremicrobiome.py; the OTU table was collapsed by cow id using collapse_samples.py, and then core successional OTUs were calculated as OTUs present in 80% of all cows. Overall, 2544 core OTUs were found. A core heat map was built using the heatmap.2 command in R. OTU table rows (bacterial species, n=2544) were clustered using hclust, and columns (rumen samples, n=1634) were sorted by day of life (see FIG. 3Aii for more details).

Core Successional Microbes Appearance in Different Dietary Regimes

To examine whether bacterial species tend to be diet-specific, we performed a permutation test (n=100) in which each row was shuffled at each iteration. Thus, the labels comprising each row were changed for each iteration, randomizing time and diet.

First Appearance of Core Successional Microbes Vs. All Other Microbes

A permutation test was performed in which the mean first presence of the core OTUs was measured. Then 2544 OTUs were randomly selected from a list of all other OTUs (core OTUs excluded) and their first presence was averaged. This step was repeated 1000 times.

Species Arrival Rate

A permutation test was performed in which the arrival of new OTUs into each time bin was measured vs. a null model. The null model was created by random shuffling of the time bin labels. This step was repeated 1000 times. The slope was measured for the non-permuted data using a linear regression model (−74) and averaged across all permutations (−134±0.5).

Persistence Analysis

Species persistence was calculated as follows. For each sampling day, the number of species arriving on that day Was counted and their maximal possible time of appearance within a window of 600 days starting from their day of first appearance was measured (Daylast appearance−Dayfirst appearance). The mean time of appearance for each sampling day was then averaged. This calculation was performed separately on core OTUs and all other OTUs. OTUs appearing later than 430 days of life were discarded due to the lower sampling depth at these time points. This analysis was repeated on OTUs appearing in at least 5, 10, 20 and 30 samples, and the same results were received.

Statistical analysis for persistence of core OTUs vs. all other OTUs was performed as follows: delta (time of last appearance−time of first appearance) was calculated for each mu, after which sampling days were shuffled and the delta recalculated 100 times. The ratio between real delta and mean permuted delta was calculated. A t-test compared the ratio between core OTUs (0.86) and all other OTUs (0.64) and was found to be significant (P<0.005, two sided).

Unique Species at First Time Point

To test for a significant association between mode of delivery and species appearance on the first day of sampling, a chi square test was performed in which the null hypothesis was that there is no relationship between species appearing at time point t=1 and mode of delivery. A contingency table was used in which all species arriving at t=1 (n=5067) were included. This table had three columns: the first column was the sum of all species unique to C-section animals (n=1163), the second was the sum of all shared species (n=1665), and the third was the sum for all species unique to vaginally delivered animals. The test rejected HO, meaning that there is a dependence between mode of delivery and species arriving at time point t=1 (P<0.05).

Phylum-Distribution Analysis

The distribution of the four main phyla (Actinobacteria, Bacteroidetes, Firmicutes, Proteobacteria) was calculated by counting the number of OTUs belonging to each of these phyla within each time bin. The difference between the distribution of each phylum between the two modes of delivery was determined by comparing the COM between the two modes of delivery by randomly sampling 70% of the values independently for C-section and vaginal delivery and calculating COM (n=1000 times). We then compared the two vectors for each phylum (1000 COM values for C-section and 1000 COM values for vaginal delivery) using Wilcoxon test.

The kernel density plot was used to present the distribution of the data (FIG. 3A). Kernel density is a weighting function that quantifies the density of samples and presents them in a smooth manner⁶⁹. The kernel density was used in order to present a histogram of the density of different phyla along time. Kernel smoothing estimates were applied to each subpopulation (C-section and vaginal delivery) and presented only the four main phyla (Actinobacteria, Bacteroidetes, Firmicutes and proteobacteria). In Kernel density, Areas with greater point density, in this case higher density of specific phylum, will have higher kernel estimate values at a specific time point, as can be seen in FIG. 3A.

COM

COM is the average time of appearance for an OTU, weighted according to its relative abundance across all sampling points. COM was calculated for each OTU as:

$$COM = \frac{{\sum }_{i = 1}^{n} R . {A}_{t} \cdot {Day}_{t}}{{\sum }_{i = 1}^{n} {Day}_{t}}$$

where i is the cow ID (1-45), R.An is the relative abundance of a species at time point t, Dayt is the day of life when the sample was taken (1-831).

MTV-LMM

MTV-LMM uses a linear mixed model for identifying autoregressive taxa and predictioning their relative abundance at future time points (see Shenhav et al. 2019 for more details). MTV-LMM is motivated by the assumption that the temporal changes in the relative abundance of taxa j are a time-homogeneous high-order Markov process. MTV-LMM models the transitions of this Markov process by fitting a sequential linear mixed model (LMM) to predict the relative abundance of taxa at a given time point, given the microbial community composition at previous time points. Intuitively, the linear mixed model correlates the similarity between the microbial community composition across different time points with the similarity of the taxa relative abundance at the next time points. MTV-LMM is making use of two types of input data: (1) continuous relative abundance of focal taxa j at previous time points and (2) quantile-binned relative abundance of the rest of the microbial community at previous time points. The output of MTV-LMM is prediction of continuous relative abundance, for each taxon, at future time points.

In order to apply linear mixed models, MTV-LMM generates a temporal kinship matrix, which represents the similarity between every pair of samples across time, where a sample is a normalization of taxa relative abundances at a given time point for a given individual. When predicting the relative abundance of taxa j at time t, the model uses both the global state of the entire microbial community in the last q time points, as well as the relative abundance of taxa j in the previous p time points. The parameters p and q are determined by the user, or can be determined using a cross-validation approach; a more formal description of their role is provided in the Methods. MTV-LMM has the advantage of increased power due to a low number of parameters coupled with an inherent regularization mechanism, similar in essence to the widely used ridge regularization, which provides a natural interpretation of the model.

Training and Testing the Model

We divide our dataset into three parts: training, validation, and testing, where each part is approximately ⅓ of the time series (sequentially). We train all four models presented above and use the validation set to select a model for each taxon j, based on the highest correlation with the real relative abundance. Using the validation set, we found p=1 and q=1 to be the best model for most taxa and therefore used these parameters. We then compute sequential out-of-sample predictions on the test set with the selected model.

Quality Control for Sequencing Errors

We measured the probability of each OTU in our data set to arise from such sequencing errors. Using a method reported earlier⁷⁰ we calculated Poisson probabilities for a single sequence at different similarities for each OTU, considering sequencing error rate of 0.24% as measured for illumina amplicon sequencing⁷¹. We defined minor and major OTUs as described in the above method and assessed whether they are artifacts arising from sequencing errors. The probability of each minor OTU arising by sequencing error was determined by multiplying this probability with the number of nucleotides in a given biological sample (which in our case is 250 nucleotides). We next tested for random sequencing error hypothesis for each ‘major’ and ‘minor’ OTUs-species (97% clusters). More than 92% and 94% respectively of the OTUs rejected the null hypothesis (using BH multiple hypotheses adjustment). Using the Bonferroni multiple hypotheses adjustment, more than 84% of the OTUs rejected the null hypothesis for a sequencing error.

Quality Control for Spurious Taxa

We examined whether of the OTUs presented in FIG. 2B as grey dots (non-core OTUs) are spurious OTUs. Using the analysis mentioned above we calculated the probability of the “grey” OTUs to be the result of sequencing error and found them all to be genuine and to reject the null hypothesis. in addition to this analysis we have measured the robustness of our results by gradually increasing the stringency of our analysis and measuring the validity and robustness of our findings. This analysis resulted in the same conclusions, further strengthening the robustness of our findings.

Availability of Data and Materials

All sequencing files and metadata are deposited in SRA under PRJNA591750.

Results

In this study, the main goal was to reveal the forces that act during the processes of rumen microbiome succession. To that end, a specific and substantial ecological perturbation at the beginning of life was introduced, consisting of two modes of delivery: C-section and vaginal delivery. The present inventors then compared the rumen microbiome assembly of these cohorts, which had uniform dietary regimes, rearing conditions and very similar genetic backgrounds (all animals were Holstein Friesian breed and genotyped). This was based on the assumption that the mode of delivery induces two distinct microbiome-composition starting points that would allow for the study of characteristics of the microbial community dynamics throughout life in light of this early perturbation^{23,24,30}. A time-series experimental setup was designed with a high sampling resolution of over 1600 samples, consisting of 45 animals, 27 born via vaginal delivery and 18 via C-section (FIG. 1A). The present inventors followed the development of their ruminal microbial community for up to 830 days (all animals except one were sampled for at least 8 months of age, and a third of the cohort was sampled over a 3-year period, with an average and standard deviation of 36±18 samples per animal, respectively). The animals were housed together from the third month of life and kept under similar conditions throughout life (FIG. 1A). During each dietary period, the animals were fed with standard dairy feeding protocols according to their age. As the animals were fed consistently with the same diets over long periods, the sampling regime consisted of multiple sampling during each dietary period, thereby enabling the inventors to distinguish diet and age effects. The focus of the study was understanding microbial species establishment and persistence in the rumen ecosystem, as well as on the forces that govern the microbial succession process.

Whereas rumen microbial composition has been previously associated with both diet and age^{18,32-34}, the relative contribution of each of these factors is still elusive. The present high-resolution sampling over time enabled the present inventors to distinguish between diet- and age-dependent effects. The analysis showed that both diet and age exert a pronounced effect on microbiome composition (FIG. 1B; PCoA based on Bray-Curtis metrics; PERMANOVA, F_{age}=20, F_{diet}=6.7, F_{diet}(F_{age}) interaction=3.7, P<0.001), with no differentiation between females and males or any effect of gestation on microbiome assembly. To disentangle the diet×age interaction effect, the inventors focused on temporal changes within specific dietary periods. As animals were kept homogeneous with respect to age and diet, it was possible to capture the direct age-dependent effects. This was mainly apparent during two stages of life, in which a constant diet was administered to the animals: during the first 2 months, when the animals were consuming milk and a starter mixture (Diet B), and during the adult stage, when the animals were nearly 3 years of age and consumed a low-fiber diet (Diet D, FIG. 1B, i and ii). Within these dietary periods, a clear and significant clustering according to age was detected (PERMANOVA, P<0.001, in both Diets B and D). Moreover, it was shown that it was possible to accurately predict the sampling time (age, as month of sampling) based on microbiome composition within each of these diets by applying a random forest classifier (accuracy levels: 0.91 for Diet B and 0.86 for Diet D;). Repetitive and consistent dynamic microbiome patterns were detected by the classifier, which were independent of diet. These diet-independent patterns were also consistent with the changes in the community alpha- and beta-diversity indices, observed during these time periods).

When examining the microbial composition between the different diets (FIG. 2A), the Bacteroidetes phylum was more dominant during the first month of life. The Bacteroidaceae was the dominant family of this phylum during the first days of life, where the calves were fed colostrum and not supplied with solid feed. Upon introduction of fiber-based diet, the Prevotellaceae family increased in relative abundance and became the most dominant family of the Bacteroidetes phylum (FIG. 2B).

As the animals progress with age and diet, from the starter mixture diet to a low-fiber diet at ˜3 months of age, a decline of the Proteobacteria phylum (mainly Succinivibrionaceae) is seen, followed by an increase of methanogenic archaeal families (Methanobacteriaceae), possibly due to the increase in fiber content (FIG. 2C). It may be speculated that these two events may be interconnected, as several studies have described a negative relationship between these two microbial families^{11,32,35,36}.

Another aspect characterizing the transition from early to adult stages is the decline of the Verrucomicrobiaceae family from 40% to 2-3% at the age of one month of the animal and its persistence at this low level of relative abundance until the end of the sampling period. Interestingly a large portion of this family's abundance is attributed to the species Akkermansia muciniphila (FIG. 2D). A. muciniphila is a mucin degrader and an abundant member of the human gut microbiome^{39-41,42}.

The transition from the first to the second month of life was followed by an increase in members of the Firmicutes phylum, which was mainly attributed to the Lachnospiraceae family (FIG. 2E), the relative abundance of which was greatly elevated. This microbial family consists of fiber degraders⁴³, yet it is unclear why its relative abundance was elevated without any dietary change. This finding would suggest that other environmental factors, driven by microbial niche modification or host control, are involved. Another family of fiber degraders is Ruminococcaceae⁴⁴. Although it has been reported that fiber degraders originating from this family are less affected by diet³², it can be clearly seen that the transition to higher fiber diet is accompanied by elevated levels of this family (FIG. 2E). In agreement with previous studies^{5,18,45,46}, members of these fiber-degrading families appear on the first day of life and increase in relative abundance before the consumption of plant fiber diet. This could suggest a potential inoculation and maintenance mechanism of the rumen ecosystem with these seminal microbial families.

A Set of Core Successional Microbes Drives Temporal Microbiome Dynamics

The recurring and consistent pattern of diet- and age-dependent clustering during microbiome development across the sampling cohort suggested that the development of the rumen microbiome is governed by microbes which can consistently be found across animals, diets and different age periods. Pursuing this hypothesis, the present inventors looked for recurring species-level microbial operational taxonomic units (OTUs) across our sample cohort (core successional microbes) and examined whether these exhibit diet- or age-dependent patterns. 2544 core successional microbes were identified, each of them present in at least 80% of the animals, in total representing most of the relative microbial abundance per animal (88% of total relative abundance). When the contribution of these core successional microbes to microbiome compositional patterns was examined, it was found that they better explained the variance and clustering patterns for age and diet than the non-core microbes. This was established by summing the R² values for age, diet and age×diet (R²_{core}=0.37) derived from a PERMANOVA on the core successional microbes and compared to 1000 iterations of random sets of non-core microbes (average R2_{non-core}=0.08).

Looking at the distribution of the core successional microbes over time (FIG. 3Ai), it was found that they tend to cluster in an age-dependent manner. More specifically, three separate clusters representing three age periods were identified (FIG. 3Ai): (a) the first month of life (days 1-30), (b) the second and third months of life (days 30-100) and (c) the fourth month to third year of life (days 100-830). Moreover, different dietary regimes were fed within these time clusters (FIG. 3Ai). Thus, when the present inventors tested for an association of the core successional microbes with specific diets, it was found that 72% of these core successional microbes appear in most diets. This result indicated that most of the core successional microbes are not diet-specific, and are associated with animal development.

Core Successional Microbiome is Acquired at Early Stages and Persists Throughout Life

The dynamic nature of the rumen microbiome composition over time raises questions regarding the forces allowing the core successional microbes to occupy multiple individuals and to persist in this ecosystem. The present inventors first sought to characterize each microbial species arrival/invasion time and its persistence during rumen microbiome succession (FIG. 3B). A significant characteristic of the core successional microbiome was identified: these core successional microbes made their first appearance earlier than all others (FIG. 3B, P<0.05, permutation test, see Methods: Persistence analysis). Furthermore, core successional microbes were found to arrive only within the first 140 days of life, with most of them introduced on the very first days after birth (days 0-10) in contrast to the invasion of other microbiome species that occurred throughout the 3 years of our sampling period.

The present inventors next asked whether the time of arrival of a species is related to its persistence within the microbiome throughout life (FIG. 3B). We measured the average persistence of microbes in relation to the day of their first appearance in the rumen ecosystem (Methods: Persistence analysis). Interestingly the core successional microbes were three times more persistent than the overall microbiome members, 600 days vs. 200 days, respectively (FIG. 3B, permutation test, P<0.05). The analysis also showed that microbial persistence has a biphasic dynamics, being overall lower during the early life stages and increasing with age. At 140 days of life, a peak in microbiome persistence was observed, which decreased after this time point and finally stabilized at 200 days of life (FIG. 3B). These results were highly robust as they remained essentially the same even when we increased substantially the stringency of our analysis. Such an outcome suggests that strong ecological selection acts on the rumen microbiome in early life and decreases with time. Notably, these changes occurred independently of the consumed diet. This suggests that arrival and persistence of microbial species is not diet-dependent and could be related to age effects. Overall, these findings suggest that microbial persistence is time-dependent and that the core successional microbes that represent most of the microbiome relative abundance are early colonizers that are highly persistent within the rumen ecosystem, potentially due to better adaptation compared to the rest of the microbiome. Family-level analysis showed that the microbial profile of these core successional microbes similar to the overall microbial family profile. This is due to the fact that core successional microbes have a very high relative abundance in the rumen microbiome (88% for all core taxa). When the present inventors examined the 10 most abundant core successional microbes, it was observed that half of them belonged to the Firmicutes phylum, all of which belonged to the Clostridiales order. Within this order, species belonging to the Shuttleworthia genus and the Ruminococcaceae family were identified. The other three species from the Firmicutes phylum were not annotated beyond the order level. Of the other 5 most abundant species, 3 belonged to the Bacteroidales order, two of them were classified as Bacteroides (genus) and one was Prevotella (genus). Another abundant core successional species belonged to the Succinivibrionaceae family, which was found to be highly dominant before the transition into high-fiber diets. Surprisingly, the A. muciniphila species was also found to be one of the 10 abundant core successional species, further highlighting this species as an important member of the rumen successional process.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

### REFERENCES

1. Hobson, P. N. & Wallace, R. J. Microbial ecology and activities in the rumen: Part II. Crit. Rev. Microbiol. 9, 253-320 (1982).
2. Mizrahi, I. The Role of the Rumen Microbiota in Determining the Feed Efficiency of Dairy Cows. in Beneficial Microorganisms in Multicellular Life Forms (eds. Rosenberg, E. & Gophna, U.) 203-210 (Springer Berlin Heidelberg, 2011).
3. Mizrahi, I. Rumen Symbioses. in The Prokaryotes: Prokaryotic Biology and Symbiotic Associations (eds. Rosenberg, E., DeLong, E. F., Lory, S., Stackebrandt, E. & Thompson, F.) 533-544 (Springer Berlin Heidelberg, 2013).
4. Huws, S. A. et al. Addressing Global Ruminant Agricultural Challenges Through Understanding the Rumen Microbiome: Past, Present, and Future. Front. Microbiol. 9, 2161 (2018).
5. Morals, S. & Mizrahi, I. Islands in the stream: From individual to communal fiber degradation in the rumen ecosystem. FEMS Microbiol. Rev. (2019) doi:10.1093/femsre/fuz007.
6. Morals, S. & Mizrahi, I. The Road Not Taken: The Rumen Microbiome, Functional Groups, and Community States. Trends Microbiol. 0, (2019).
7. Weimer, P. J., Russell, J. B. & Muck, R. E. Lessons from the cow: what the ruminant animal can teach us about consolidated bioprocessing of cellulosic biomass. Bioresour. Technol. 100, 5323-5331 (2009).
8. Xue, M., Sun, H., Wu, X., Guan, L. L. & Liu, J. Assessment of Rumen Microbiota from a Large Dairy Cattle Cohort Reveals the Pan and Core Bacteriomes Contributing to Varied Phenotypes. Appl. Environ. Microbiol. 84, (2018).
9. Skarlupka, J. H., Kamenetsky, M. E., Jewell, K. A. & Suen, G. The ruminal bacterial community in lactating dairy cows has limited variation on a day-to-day basis. J. Anim. Sci. Biotechnol. 10, 66 (2019).
10. Sasson, G., Ben-Shabat, S. K. & Seroussi, E. Heritable Bovine Rumen Bacteria Are Phylogenetically Related and Correlated with the Cow's Capacity To Harvest Energy from Its Feed. MBio (2017).
11. Wallace, R. J. et al. A heritable subset of the core rumen microbiome dictates dairy cow productivity and emissions. Sci Adv 5, eaav8391 (2019).
12. Ramayo-Caldas, Y. et al. Identification of rumen microbial biomarkers linked to methane emission in Holstein dairy cows. J. Anim. Breed. Genet. (2019) doi:10.1111/jbg.12427.
13. Lima, J. et al. Identification of Rumen Microbial Genes Involved in Pathways Linked to Appetite, Growth, and Feed Conversion Efficiency in Cattle. Front. Genet. 10, 701 (2019).
14. Shabat, S. K. B. et al. Specific microbiome-dependent mechanisms underlie the energy harvest efficiency of ruminants. ISME J. 10, 2958-2972 (2016).
15. Zhou, J. & Ning, D. Stochastic Community Assembly: Does It Matter in Microbial Ecology? Microbiol. Mol. Biol. Rev. 81, (2017).
16. Fukami, T. Historical Contingency in Community Assembly: Integrating Niches, Species Pools, and Priority Effects. Annu. Rev. Ecol. Evol. Syst. 46, 1-23 (2015).
17. Martínez, I. et al. Experimental evaluation of the importance of colonization history in early-life gut microbiota assembly. Elife 7, (2018).
18. Jami, E., Israel, A., Kotser, A. & Mizrahi, I. Exploring the bovine rumen bacterial community from birth to adulthood. ISME J. 7, 1069-1079 (2013).
19. Costello, E. K., Stagaman, K., Dethlefsen, L., Bohannan, B. J. M. & Relman, D. A. The application of ecological theory toward an understanding of the human microbiome. Science 336, 1255-1262 (2012).
20. Stewart, C. J. et al. Cesarean or Vaginal Birth Does Not Impact the Longitudinal Development of the Gut Microbiome in a Cohort of Exclusively Preterm Infants. Front. Microbiol. 8, 1008
21. Chu, D. M. et al. Maturation of the infant microbiome community structure and function across multiple body sites and in relation to mode of delivery. Nat. Med. 23, 314-326 (2017).
22. Bokulich, N. A. et al. Antibiotics, birth mode, and diet shape microbiome maturation during early life. Sci. Transl. Med. 8, 343ra82 (2016).
23. Yassour, M. et al. Natural history of the infant gut microbiome and impact of antibiotic treatment on bacterial strain diversity and stability. Sci. Transl. Med. 8, 343ra81 (2016).
24. Bäckhed, F. et al. Dynamics and Stabilization of the Human Gut Microbiome during the First Year of Life. Cell Host Microbe 17, 690-703 (2015).
25. Shao, Y. et al. Stunted microbiota and opportunistic pathogen colonization in caesarean-section birth. Nature (2019) doi:10.1038/s41586-019-1560-1.
26. Hansen, C. H. F. et al. Mode of delivery shapes gut colonization pattern and modulates regulatory immunity in mice. J. Immunol. 193, 1213-1222 (2014).
27. Frutos, J. et al. Early feed restriction of lambs modifies ileal epimural microbiota and affects immunity parameters during the fattening period. Animal 12, 2115-2122 (2018).
28. Saro, C. et al. Effectiveness of Interventions to Modulate the Rumen Microbiota Composition and Function in Pre-ruminant and Ruminant Lambs. Front. Microbiol. 9, 1273 (2018).
29. Abecia, L. et al. Natural and artificial feeding management before weaning promote different rumen microbial colonization but not differences in gene expression levels at the rumen epithelium of newborn goats. PLoS One 12, e0182235 (2017).
30. Dominguez-Bello, M. G. et al. Delivery mode shapes the acquisition and structure of the initial microbiota across multiple body habitats in newborns. Proc. Natl. Acad. Sci. U.S.A 107, 11971-11975 (2010).
31. Surlis, C. et al. Birth delivery method affects expression of immune genes in lung and jejunum tissue of neonatal beef calves. BMC Vet. Res. 13, 391 (2017).
32. Henderson, G. et al. Rumen microbial community composition varies with diet and host, but a core microbiome is found across a wide geographical range. Sci. Rep. 5, 14567 (2015).
33. Dill-McFarland, K. A., Weimer, P. J., Breaker, J. D. & Suen, G. Diet Influences Early Microbiota Development in Dairy Calves without Long-Term Impacts on Milk Production. Appl. Environ. Microbiol. 85, e02141-18 (2019).
34. Wang, L. et al. Exploring the Goat Rumen Microbiome from Seven Days to Two Years. PLoS One 11, e0154354 (2016).
35. Pope, P. B. et al. Adaptation to herbivory by the Tammar wallaby includes bacterial and glycoside hydrolase profiles different from other herbivores. Proc. Natl. Acad. Sci. U.S.A 107, 14793-14798 (2010).
36. Wallace, R. J. et al. The rumen microbial metagenome associated with high methane production in cattle. BMC Genomics 16, 839 (2015).
37. He, J. et al. Characterizing the bacterial microbiota in different gastrointestinal tract segments of the Bactrian camel. Sci. Rep. 8, 654 (2018).
38. Wang, L. et al. Bacterial Community Diversity Associated With Different Utilization Efficiencies of Nitrogen in the Gastrointestinal Tract of Goats. Front. Microbiol. 10, 239 (2019).
39. Michalovich, D. et al. Obesity and disease severity magnify disturbed microbiome-immune interactions in asthma patients. Nat. Commun. 10, 5711 (2019).
40. Presti, A. L. et al. Exploring the genetic diversity of the 16S rRNA gene of Akkermansia muciniphila in IBD and IBS. Future Microbiol. 14, 1497-1509 (2019).
41. Lawenius, L. et al. Pasteurized Akkermansia muciniphila protects from fat mass gain but not from bone loss. Am. J. Physiol. Endocrinol. Metab. (2020) doi:10.1152/ajpendo.00425.2019.
42. Derrien, M., Collado, M. C., Ben-Amor, K., Salminen, S. & de Vos, W. M. The Mucin degrader Akkermansia muciniphila is an abundant resident of the human intestinal tract. Appl. Environ. Microbiol. 74, 1646-1648 (2008).
43. Deusch, S. et al. A Structural and Functional Elucidation of the Rumen Microbiome Influenced by Various Diets and Microenvironments. Front. Microbiol. 8, 1605 (2017).
44. Vos, P. et al. Bergey's Manual of Systematic Bacteriology: Volume 3: The Firmicutes. (Springer Science & Business Media, 2011).
45. Guzman, C. E., Bereza-Malcolm, L. T., De Groef, B. & Franks, A. E. Presence of Selected Methanogens, Fibrolytic Bacteria, and Proteobacteria in the Gastrointestinal Tract of Neonatal Dairy Calves from Birth to 72 Hours. PLoS One 10, e0133048 (2015).
46. Minato, H., Otsuka, M., Shirasaka, S., Itabashi, H. & Mitsumori, M. COLONIZATION OF MICROORGANISMS IN THE RUMEN OF YOUNG CALVES. J. Gen. Appl. Microbiol. 38, 447-456 (1992).
47. Dufrene, M. & Legendre, P. Species Assemblages and Indicator Species: The Need for a Flexible Asymmetrical Approach. Ecological Monographs vol. 67 345 (1997).
48. Shenhav, L. et al. Modeling the temporal dynamics of the gut microbial community in adults and infants. doi:10.1101/212993.
49. Mamun, M. A. A. et al. The composition and stability of the faecal microbiota of Merino sheep. J. Appl. Microbiol. (2019) doi:10.1111/jam.14468.
50. Zhou, F. et al. Dietary Bovine Milk Exosomes Elicit Changes in Bacterial Communities in C57BL/6 Mice. Am. J. Physiol. Gastrointest. Liver Physiol. (2019) doi:10.1152/ajpgi.00160.2019.
51. Robertson, S. J. et al. Comparison of Co-housing and Littermate Methods for Microbiota Standardization in Mouse Models. Cell Rep. 27, 1910-1919.e2 (2019).
52. Garrido, D. et al. Utilization of galactooligosaccharides by Bifidobacterium longum subsp. infantis isolates. Food Microbiol. 33, 262-270 (2013).
53. Gomez-Gallego, C. et al. Resembling breast milk: influence of polyamine-supplemented formula on neonatal BALB/cOlaHsd mouse microbiota. Br. J. Nutr. 111, 1050-1058 (2014).
54. Geerlings, S. Y., Kostopoulos, I., de Vos, W. M. & Belzer, C Akkermansia muciniphila in the Human Gastrointestinal Tract: When, Where, and How? Microorganisms 6, (2018).
55. Li, F. & Guan, L. L. Metatranscriptomic Profiling Reveals Linkages between the Active Rumen Microbiome and Feed Efficiency in Beef Cattle. Appl. Environ. Microbiol. 83, (2017).
56. Rey, M. et al. Establishment of ruminal bacterial community in dairy calves from birth to weaning is sequential. J. Appl. Microbiol. 116, 245-257 (2014).
57. Avgustin, G., Wallace, R. J. & Flint, H. J. Phenotypic Diversity among Ruminal Isolates of Prevotella ruminicola: Proposal of Prevotella brevis sp. nov., Prevotella bryantii sp. nov., and Prevotella albensis sp. nov. and Redefinition of Prevotella ruminicola. Int. J. Syst. Bacteriol. 47, 284-288 (1997).
58. Purushe, J. et al. Comparative genome analysis of Prevotella ruminicola and Prevotella bryantii: insights into their environmental niche. Microb. Ecol. 60, 721-729 (2010).
59. Charbonneau, M. R. et al. Sialylated Milk Oligosaccharides Promote Microbiota-Dependent Growth in Models of Infant Undernutrition. Cell 164, 859-871 (2016).
60. Korpela, K. et al. Selective maternal seeding and environment shape the human gut microbiome. Genome Res. 28, 561-568 (2018).
61. Kokou, F. et al. Core gut microbial communities are maintained by beneficial interactions and strain variability in fish. Nat Microbiol (2019) doi:10.1038/s41564-019-0560-0.
62. Shaani, Y., Zehavi, T., Eyal, S., Miron, J. & Mizrahi, I. Microbiome niche modification drives diurnal rumen community assembly, overpowering individual variability and diet effects. ISME J. (2018) doi:10.1038/s41396-018-0203-0.
63. Kolkman, I. et al. Protocol of the Caesarean section as performed in daily bovine practice in Belgium. Reprod. Domest. Anim. 42, 583-589 (2007).
64. Stevenson, D. M. & Weimer, P. J. Dominance of Prevotella and low abundance of classical ruminal bacterial species in the bovine rumen revealed by relative quantification real-time PCR. Appl. Microbiol. Biotechnol. 75, 165-174 (2007).
65. Dehority, B. A. & Grubb, J. A. Effect of short-term chilling of rumen contents on viable bacterial numbers. Appl. Environ. Microbiol. 39, 376-381 (1980).
66. Purcell, S. et al. PLINK: a tool set for whole-genome association and population-based linkage analyses. Am. J. Hum. Genet. 81, 559-575 (2007).
67. Kang, H. M. Efficient Mixed-Model Association eXpediated (EMMAX). UCLA Law Rev. (2010).
68. Caporaso, J. G. et al. QIIME allows analysis of high-throughput community sequencing data. Nat. Methods 7, 335-336 (2010).
69. He, H., Wang, W. & Tang, W. Prediction model-based kernel density estimation when group membership is subject to missing. Adv Stat Anal 101, 267-288 (2017).
70. Jing, X. et al. The bacterial communities in plant phloem-sap-feeding insects. Molecular Ecology vol. 23 1433-1444 (2014).
71. Pfeiffer, F. et al. Systematic evaluation of error rates and causes in short samples in next-generation sequencing. Sci. Rep. 8, 10950 (2018).

1. (canceled)

2. A method of improving a commercially desirable phenotype of an adult ruminant comprising administering to the ruminant when it is at the newborn stage of life, a composition which alters the amount of the genus Akkermansia in the microbiome of the newborn ruminant, wherein said composition is:
(i) a microbial composition, wherein at least 5% of the microbes of the composition belong to the genus Akkermansia;
(ii) an antibiotic which specifically targets said genus Akkermansia; and/or
(iii) a prebiotic or dietary ingredient which alters the amount of Akkermansia in said newborn, thereby improving a commercially desirable phenotype of an adult ruminant.

3. A method of selecting an agent which improves a commercially desirable phenotype of an adult ruminant comprising:
(a) administering to the ruminant when it is at the newborn stage of life, a composition comprising the agent which alters the amount of the genus Akkermansia in the microbiome of the newborn ruminant; and
(b) analyzing the desirable phenotype in the ruminant at the adult stage of life, when an improvement of said desirable phenotype is indicative that said agent has a positive effect on said desirable phenotype.

4. The method of claim 3, wherein the agent is selected from the group consisting of:
(i) a microbe;
(ii) an antibiotic which specifically targets said genus Akkermansia; and
(iii) a prebiotic or dietary ingredient which alters the amount of Akkermansia in said newborn.

5. The method of claim 2, wherein said newborn stage of life is younger than 15 days.

6. The method of claim 2, wherein said bacteria comprises the species Akkermansia muciniphila.

7-8. (canceled)

9. The method of claim 2, wherein said microbiome comprises the rumen microbiome.

10. The method of claim 2, wherein the commercially desirable phenotype is selected from the group consisting of an increase in fertility, a decrease in the propensity to infection, a decrease in methane production, an increase in milk production, an increase in milk quality, an increase in meat quality and an increase in feed efficiency.

11. The method of claim 10, wherein said milk quality is selected from the group consisting of a fat content, a lactose content and a protein content.

12. The method of claim 10, wherein the infection is selected from the group consisting of brucellosis, campylobacteriosis, cryptosporidiosis, mastitis, Escherichia coli 0157:H7, Q Fever (Coxiella burnetti) infection and Salmonella infection.

13. The method of claim 2, wherein said administering is effected more than one time.

14. The method of claim 2, wherein said composition is comprised in a feed, a silage or an enema.

15-16. (canceled)

17. The method of claim 2, further comprising administering to the ruminant an antibiotic prior to the administration of the agent.

18-19. (canceled)

20. The method of claim 2, wherein said ruminant is not weaned.

21. A microbial composition comprising a plurality of microbes, wherein at least 10% of the microbes are Akkermansia muciniphila.

22. The microbial composition of claim 21, being formulated as an enema.

23. The microbial composition of claim 21, further comprising bacteria of the Succinivibrionaceae family, the Lachnospiraceae family and/or the Ruminococcus genus.

24. A feed comprising the microbial composition of claim 21.


SEQUENCE LISTING (176 sequences)

SEQ ID NO: 1 - DNA, 253 bp, Akkermansia
tacagagatctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 2 - DNA, 253 bp, Akkermansia
tacagagggtgcaagcgttaatcggaattactgggcgtaaagcgagcgtaggtggcttgataagtcagatgtgaaagccccgggcttaacctgggaacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 3 - DNA, 251 bp, Akkermansia
tacagaggtcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 4 - DNA, 253 bp, Akkermansia
tacagaggtctcaagcgttgttcggaatcactgggcgtaaagcatgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 5 - DNA, 253 bp, Akkermansia
tacagaggtctcaagcgttgttcggaatcactgggcgtaaagcgtgcgtaggcggtttcgtaagtcgtgtgtgaaaggcgggggctcaacccccggactgcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 6 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaaatctcggtgtagcagtgaaatgcgtagatatcgagaggaacactcgtggcgaaggcgggttcctggacattaactgacgctgaggcacgaaggccaggggagcgaaaggg

SEQ ID NO: 7 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattcccagtgtagcggtgaaatgcgtagatattgggaggaacaccagtggcgaaggcgcctttctggactgtgtctgacgctgaggagcgaaagcgtggggagcgaacagg

SEQ ID NO: 8 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattcccggtgtagcggtgaaatgcgtagatattaggaagaacaccagtggcgaaggcggctttctggacgacaactgacgctgaggcgcgaaagccaggggagcgaacggg

SEQ ID NO: 9 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattcccggtgtagcggtggaatgcgtagatatcgggaggaacaccagtggcgaaggcggcctactgggcactaactgacgctgaggctcgaaagccaggggagcgaacggg

SEQ ID NO: 10 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattcctagtgtagcggtgaaatgctcagatatcatgaggaactccaatcgcgaaggcaggcatccgggccggaactgacactgaggctcgaaagtgcgggtatcaaacagg

SEQ ID NO: 11 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattcgcggtgtagcagtgaaatgcgtagatatcaggaggaacaccaatggcgaaggcaggtctctgggcagtaactgacgctgaggagcgaaagcatgggtagcgaacagg

SEQ ID NO: 12 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattcgcggtgtagcagtgaaatgcgtagatatcgagaggaacactcgtggcgaaggcgggttcctggacattaactgacgctgaggcacgaaggccaggggagcgaaaggg

SEQ ID NO: 13 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggcgtagcagtgaaatgcgtagatatcgagaggaacactcgtggcgaaggcgggttcctggacattaactgacgctgaggcacgaaggccaggggagcgaaaggg

SEQ ID NO: 14 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagagatggggaggaacaccagtggcgaaggcggcctgctgggctttaactgacgctgaggcacgaaagcgtgggtagcaaacagg

SEQ ID NO: 15 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagagatgtgaaggaataccgaaggcgaaggcagccccttgggaagatactgacgctcatgtgcgaaagcgtggggagcaaacagg

SEQ ID NO: 16 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatacggaggaacaccagtggcgaaggcggcctgctggacattaactgacgctgaggcgcgaaagcgtggggagcaaacagg

SEQ ID NO: 17 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcacgaagaactcctattgcgaaggcagcttgctggagtgtaactgacgctgatactcgaaagtgtgggtatcaaacagg

SEQ ID NO: 18 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacaccagtggcgaaggcggcttactggacggtaactgacgttgaggctcgaaagcgtggggagcaaacagg

SEQ ID NO: 19 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacaccagtggcgaaggtggcctactgggcactaactgacgctgaggctcgaaagcatgggtagcaaacagg

SEQ ID NO: 20 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacaccggtggcgaaggcggtcttctgggcctttactgacgctaaagtacgaaagctaggggagcaaacggc

SEQ ID NO: 21 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcggcctactgggcactaactgacgctgatgctcgaaagtgtgggtatcaaacagg

SEQ ID NO: 22 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcggctttctggactgcaactgacactgaggcacgaaagcgtggggagcaaacagg

SEQ ID NO: 23 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggtctctggtctgtaactgacgctgaggctcgaaagcgtggagagcaaacagg

SEQ ID NO: 24 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 25 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggcgaggggagcgaaaggg

SEQ ID NO: 26 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagcggaacactcgtggcgaaggcgggttcctggacattaactgacgctgaggcacgaaggccaggggagcgaaaggg

SEQ ID NO: 27 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatattaggaagaacaccagtggcgaaggcgacttactggactgaaactgacgctgaggctcgaaagcgtgggaagcaaacagg

SEQ ID NO: 28 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatattaggaggaacaccgattgcgaaggcagcttaccaaactatatctgacgttgaggcacgaaagcgtggggagcaaacagg

SEQ ID NO: 29 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatattgggaggaacaccagtggcgaaggcgcctttctggactgtgtctgacgctgagatgcgaaagccagggtagcgaacggg

SEQ ID NO: 30 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgtgcagatatcaggaggaacaccgatggcgaaggcaggtctctgggcagttactgacgctgaggagcgaaagcatgggtagcgaacagg

SEQ ID NO: 31 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgagatgcgtagatattaggaggaacaccagtggcgaaggcggattgctggacagtaactgacggtgaggcgcgagagcgtggggagcaaacagg

SEQ ID NO: 32 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcttagatattacatggaataccaattgcgaaggcaggttactaacaaacgattgacgctgatggacgaaagcgtgagtagcgaacagg

SEQ ID NO: 33 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtggaatgcgtagatatcgggaggaacaccagtggcgaaggcggcctactgggcactaactgacgctgaggctcgaaagcatgggtagcaaacagc

SEQ ID NO: 34 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgggactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 35 - DNA, 253 bp, Akkermansia
tacagaggtctcaagcgttgttcggaatcactgggcgtaaagcgtgcgtaggctgtttcgtaagtcgtgtgtgaaaggtgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 36 - DNA, 252 bp, Akkermansia
tacagaggtctcaagcgttgttcgggatcactgggcgtaaagcgtgcgtaggctgtttcgtaagtcgtgtgtgaaagcgcggggctcaaccccgtacagcggcgcgaactgctccccttggttgcgggaagggaaggcggaactcgtggtgtagcggtgaaatgcgtagatatcgagaggaacactcgtggcgaaggcgggttcctggacattaactgacgctgaggcacgaaggccaggggagcgaagggg

SEQ ID NO: 37 - DNA, 253 bp, Akkermansia
tacagaggtctcgagcgttgttcggaatcactgggcgtaaagagtgcgtaggctgtttcgtaagtcgtgtgtgaaaggcgcgggctcaacccgcggagtgcacatgatactgcgagactggagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 38 - DNA, 253 bp, Akkermansia
taccgaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 39 - DNA, 253 bp, Akkermansia
tacgaagggggctagcgttgttcggaattactgggcgtaaagcgcgcgtaggcggatcggtcagttaggggtgaaatcccggggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 40 - DNA, 253 bp, Akkermansia
tacggaggatccgagcgttatccggatttattgggtttaaagggtgcgtaggctgtttcgtaagtcgtgtgtgaaaggcgcgggcccaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 41 - DNA, 253 bp, Akkermansia
tacggaggatgcgagcgttatccggaattattgggtttaaagggagcgtaggtgggatattaagtcagctgtgaaagtttggggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 42 - DNA, 253 bp, Akkermansia
tacggaggattcaagcgttatccggatttattgggtttaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 43 - DNA, 253 bp, Akkermansia
tacggagggtgcaagcgttaatcggaattactgggcgtaaagcgcgcgtaggtggcttgataagccggttgtgaaagccccgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 44 - DNA, 253 bp, Akkermansia
tacgtagggggcaagcgttgttcggaatcactgggcgtaaagcgtgcgtaggctgtttcgtaagtcgtgtgtgaaaggcgcgagctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 45 - DNA, 253 bp, Akkermansia
tacgtagggtccgggcgttatccggatttactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 46 - DNA, 253 bp, Akkermansia
tacagaggtctcaagcgttgttcggcatccctgggcgtcaagcgtgcgtaggctgtttcgtacgtcgtgtgtgaaaggcgcgggctcagcccgcggacggcacatgatactgcgagactagagtaatggagggggaaccggaattctcggtgtagcagtgaaaggcgtagatatcgagaggaacactcgtggcgaaggcgggttcatggacattaactgacgctgaggcacgaaggtcaggggagcgaaaggg

SEQ ID NO: 47 - DNA, 253 bp, Akkermansia
tacagaggtctcaagcgttgttcggaatcactgggcgtagagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacagatactgacgctaaagcgcgaaagcgtgggtagcaaacagg

SEQ ID NO: 48 - DNA, 253 bp, Akkermansia
tacgtaggtggcaagcgttatccggatttactgggtgtaaagggcgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 49 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcaggaggaacaccggtggcgaaggcggcttactggacggcaactgacgttgaggctcgaaagcgtggggagcaaacagg

SEQ ID NO: 50 - DNA, 253 bp, Akkermansia
tacgtagggggcgagcgttgtccggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaaggcgtagatagcgagaggaacactcgtggcgaaggcgggttcctggacattaactgacgctgaggcacgaaggccaggggagcgaaaggg

SEQ ID NO: 51 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagatatcatgaagaacactcgtggcgaaggcgggtttctggacattaactgacgctgaggcacgaaagcgaggggagcgaaaagg

SEQ ID NO: 52 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgcaatgcgtagatagcgagaggagcactcggggcgaaggcggggtactggacattaaccgacgctgaggcacgaaggccagggtagcgaaaggg

SEQ ID NO: 53 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagccgtgacatgcgtagatataaggaagaacaccagtggcgaaggcggtctactggacagtaactgacgctgaggcgcgaaagcgtggggagcaaacagt

SEQ ID NO: 54 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtggagcggtgaaaagcgtagaaatcgagaggaacacgcggggcgaaggcgggttcagggacattaactgacgctgaggcacgaaggccaggggagcgaaaggg

SEQ ID NO: 55 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgggagactagagtaatggagggggagccggaattctcggtgtagcggtgaaatgcgtagatatcgagaggaacacccgtggcgaaggcaggttcctggacagtaactgacgctgatgcacgaaggcgagggtagcgaaaagg

SEQ ID NO: 56 - DNA, 253 bp, Akkermansia
tacggagggtgcgagcgttaatcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtggagcagtgaaatgcggagatatcgagaggaacacccgtggcgaaggcgggttcctggacattaactgacgctgaggcacgaaggccaggggagcgaaaggg

SEQ ID NO: 57 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcaggaggaacaccggtggcgaaggcggtcttctgggcctttactgacgctaaagtacgaaagctaggggagcaaacggg

SEQ ID NO: 58 - DNA, 253 bp, Akkermansia
tacagaggtctcaagcgttgttcggaatcactgggcgtaaagcgcgcgtaggctgtttcgtaagtcgtgtgtgaaagccgcgggctcaacccgcggacggcacatgatactgcgagactagagtaatggagggggagccggaattctcggtgtagcggtgaaatgcgtagatatcgggaggaacactcgtggcgaaggcgggttcctggacattaactgacgctgaggcgcgaaggcgaggggagcgaaaagg

SEQ ID NO: 59 - DNA, 253 bp, Akkermansia
tacagaggtctcaagcgttgttcggaatcactgggcgtaaagcgtgcgtaggctgtttcgtaggtcgtgtgtgaaaggcgcgggctcaacccgcggacggcacatgatactgcgagactagagtaatggagggggaaccggaattctcggtgtagcagtgaaatgcgtagatatatggaagaacaccagtggcgaaggcggctgtctggtctgcaactgacgctgaggctcgaaagcatgggtagcgaacagg

SEQ ID NO: 60 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtggcagggaaatgcgtagagatcgagaggaacactcggggcgaaggggggttaatggaaattaactgacgctgaggcacgaaggcgaggggagcgaaaggg

SEQ ID NO: 61 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagagatcgagaggaacactcgtggcgaaggcgggttactggacagtaactgacgcggaggcgcgaaggggaggggagcgaaaggg

SEQ ID NO: 62 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcggcttgctgggccattactgacgctgagacacgaaagcgtggggagcaaatagg

SEQ ID NO: 63 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatatggaggaacaccagtggcgaaggcgacggtctgggcctgcactgacgctcattcccgaaagcgtggggagcaaatagg

SEQ ID NO: 64 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcacgaggaactccgattgcgaaggcagcttactgtagttgtactgacgctgaagctcgaaggtgcgggtatcgaacagg

SEQ ID NO: 65 - DNA, 253 bp, Akkermansia
tacagaggtctcaagcgttgttcggaatcactgggcgtaaagcgtgcgtaggctgtttcgtaagtcgtgtgtgacaggcgcgggctcaacccgcggacggcacatgatactgcgaggctagagtaatggagggggaaccggaattctcggtgtagcagtgaaatgcgtagatatcgagaggaacactcgtggcgaaggcgggttcctggacattaactgacgctgaggctcgaaggccaggggagcaaacggg

SEQ ID NO: 66 - DNA, 253 bp, Akkermansia
tacggaggatccgagcgttatccggatttattgggtttaaagggagcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 67 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcggattgctggacagtaactgacgctgaggcgcgaaagcgtggggagcaaacagg

SEQ ID NO: 68 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagagagcgagaggaacactagtggcgaaggcgggttactggacagtaactgacgctgaggcgcgaaggcgaggggagcgaaaggg

SEQ ID NO: 69 - DNA, 253 bp, Akkermansia
tacagaggtctcaagcgttgttcggaatcactgggcgtaaagcgtgcgtcggctgtttcgtaagtcttgtgtgaaaggcgcgggctcaacccgcggactgcacatgatactgcgagactagagtaatggagggggaaccggaattctcggtgtagcggtgaaaggcgtagatatcgagaggaacactcgaggcgaaggcgggttactggacattaactgacgctgaggcacgaaggccaggggagcgaaaagg

SEQ ID NO: 70 - DNA, 253 bp, Akkermansia
tacggaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaggcggaattctcggtgtagcggtgaaatgcgtagatatcgagaggaacaccggtggcgaaggcgggttcctggacattaactgacgctgaggcacgaaggcgaggggagcgaaaagg

SEQ ID NO: 71 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtggaatgcgtagagatcgagaggaacactcgtggcgaaggcgggttcctggacattaaccgacgctgaggcacgaaggcgaggggagcgaaaggg

SEQ ID NO: 72 - DNA, 253 bp, Akkermansia
tacgtaggtggcaagcgttgtccggaattattgggcgtaaagcgcgcgcaggcggatgcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 73 - DNA, 253 bp, Akkermansia
tacgtagggcgcgagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcaaaaggg

SEQ ID NO: 74 - DNA, 253 bp, Akkermansia
tacagaggtctcaagcgttgttcggaatcactgggcgtaaagcgtgcgtaggctgtttcgtaagtcgtgtgtggaaggcgcgggctcaacccgcggacggcacatgatactgcgagactagagtaatggagggggaaccggaattctcggtgtagcggtgaaatgcgtagatatcgagaggaacactcgtggcgaaggcgggttcctggacattaactgacgctgaggcacgaaggcgaggggagcaaaaggg

SEQ ID NO: 75 - DNA, 253 bp, Akkermansia
tacagaggtctcaagcgttgttcggaatcactgggcgtaaagcgtgcgtaggctgtttcgtaagtcgtgtgtgaaaggtgcgggctcaacccgcggacggcacatgatactgcgagactagagtaatggagggggaaccggaattccttgtgtagcggtgaaatgcgtagatataaggaagaacaccagtggcgaaggcggtctactggacagtaactgacgctgaggcgcgaaagcgtggggagcaaacagg

SEQ ID NO: 76 - DNA, 253 bp, Akkermansia
tacagaggtctcaagcgttgttcggaatcactgggcgtaaagcgtgcgtaggctgtttcgtaagtcgtgtgtgaaagtcgcgggctcaacccgcggacggcacatgatactgcgagactagagtaatggagggggaaccggaattctcggtgtagcggtgaaatgcgtagatatcgggaggaacacccgtggcgaaggcgggttcctggacattaactgacgctgaggcgcgaaggccaggggagcaaaaggg

SEQ ID NO: 77 - DNA, 253 bp, Akkermansia
tacagaggtctcaagcgttgttcggaatcactgggcgtaaagcgtgcgtaggctgtttcgtaagtcgtgtgtgaaaggtgcgggctcaacccgcggacggcacatgatactgcgagactagagtaatggagggggaaccggaattctcggtgtagcggtgaaatgcgtagatatcgagaggaacactcgtggcgaaggcggcccgctggactgtaactgacgttgaggctcgaaagcgtggggagcaaacagg

SEQ ID NO: 78 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggacttctcggtgtagcagtgaaatgcgtagagatctggaggaataccgatggcgaaggcgggttcctggacattaactgacgctgaggcacgaaggccaggggagcgaaaggg

SEQ ID NO: 79 - DNA, 253 bp, Akkermansia
tacagaggtctcaagcgttgttcggaatcactgggcgtaaagcgtgcgtaggctgtttcgtaagtcgtgtgtgaaaggctcgggctcaaccctcggacggcacatgatactgcgagactagagtaatggagggggaaccggaattctcggtgtagcggtgaaatgcgtagatatcgcgaggaacacccgtggcgaaggcaggttgctggacagtaactgacgctgaggcacgaaggccaggggagcgaaaggg

SEQ ID NO: 80 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattcccggtgtagcggtgaaatgcgtagagatcgggaggaacaccagtggcgaaggcgacttactggactgtaactgacgctgaggcgcgaaagtgtggggagcaaacagg

SEQ ID NO: 81 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcggttacctggtctgtaactgacgctcaggcacgaaagcgtggggagcaaatagg

SEQ ID NO: 82 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagagatcgagaggaacactcggggcgaaggcgggtgcctggacatgaactgacgctgaggtgcgaaggcgaggggagcgaaaggg

SEQ ID NO: 83 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatattaggaggaacaccagtggcgaaggcggattgctggacagtaactgacggtgaggcgcgagagcgtggggagcaaacagg

SEQ ID NO: 84 - DNA, 253 bp, Akkermansia
tacagaggtctcaagcgttgttcggaatcactgggcgtaaagcgtgcgcaggctgtttcgtaagtcgtgtgtgaaaggcgcgggctcaacccgcggacggcacatgatactgcgagactagagtaatggagggggagccggaattctcggtgtagcggtgaaatgcgtagatatcgggaggaacacgcgaggcgaaggcgggttcttggacagtaactgacgctgaggcgcgaaggcgaggggagcgaaaggg

SEQ ID NO: 85 - DNA, 253 bp, Akkermansia
tacagaggtctcaagcgttgttcggaatcacttggcgtaaagcgtgcgtaggctgtttcgtaattcgtgtgtgaaaggcgcgggctcaacccgcggacggcacatgatactgcgagactagagtaatggagggggaaccggaattctcggtgtagaagtgaaatgcgtagatatcgagaggaacaagcgtggcgaaggagggttcctggacattaactgacgctgaggcacgaaggtgaggggagcgaaaggg

SEQ ID NO: 86 - DNA, 253 bp, Akkermansia
tacagaggtctcaagcgttgttcggaatcactgggcgtaaagtgtgcgtaggctgtttcgtaagtcgtgtgtgaaaggcgcgggctcaacccgcggacggcacatgatactgcgagactagagtaatggagggggaaccggaattccatgtgtagcggtgaaatgcgtagatatatggaggaacaccagtggcgaaggcggctttctggtctgtaactgacgctgaggctcgaaagcgtggggagcaaacagg

SEQ ID NO: 87 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagatatcgagaggaacactcgtggcgaaggcgggttgctggacattaactgacgttgaggctcgaaagcgtggggagcgaacagg

SEQ ID NO: 88 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggccggcacatgatactgcgagactagagtaatggagggggaaccggaattctcggtgtagcggtgaaatgcgtagatatcgggaggaacactcgtggcgaaggcgggttcctggacattaactgacgctgaggcacgaaggcgaggggagcaaaaagg

SEQ ID NO: 89 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatagcgagaggaagactcgtggcgagggcgagtgcctggacattaacggacgctgaggcacgaaggccaggggagcgaaaggg

SEQ ID NO: 90 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagacttgagtaatggagggggaaccggaattctcggtgtagcagtgaaatgcgtagatatcgagaggaacactcgtggcgaaggcgggtgccgggacattaactgacgctgaggcacgaaagcgaggggatcgaacagg

SEQ ID NO: 91 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtggcggtgacatgcgtagatatcaagaggaacacacgtggcgaaggcgggttcctggacattaactgacgctgaggcccgaaggcgaggggagcgaaagga

SEQ ID NO: 92 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagatatcgagaggaacacacgtggcgaaggcaggtgcatagacattaactgacgctgaggcgcgaaggcgaggggagcgaaaggg

SEQ ID NO: 93 - DNA, 253 bp, Akkermansia
tacagaggtctcaagcgttgttcggaatcactgggcgtcaagcgtgcgtaggctgtttcgtaagtcgtgtgtgacaggcgcgggctcaacccgcggacggcacaggatactgcgagactagagtaatggagggggaaccggaattctcggtgtagcggggaaatgcgtagagatcgagaggaacactcgtggcgaaggcgggtgcctggacattaactgacgctgaggcacgaaagccaggggagcgaaaggg

SEQ ID NO: 94 - DNA, 253 bp, Akkermansia
tacagaggtcttaagcgttgttcggaatcactgggcgtaaagcgtgcgtaggctgtttcgtaagtcgtgtgtgaaaggcgcgggctcaacccgcggccggcacatgatactgcgagactagagtaatggagggggaaccggaattctcggtgtagcggtggaatgcgtagatatcgggaggaacactcgtggcgaaggcgggttcctgggcattgactgacgctgaggcgcgaaagctaggggagcgaaaggg

SEQ ID NO: 95 - DNA, 253 bp, Akkermansia
tacggagggcgcgagcgttgtccggaattattgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 96 - DNA, 253 bp, Akkermansia
tacagaggtctcaagcgttgttcggaatcactgggcgtaaagcgtgcgtaggctgtttcgtacgtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcggcctgctgggcttttactgacgctgaggctcgaaagtgtggggagcaaacagg

SEQ ID NO: 97 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcggcctactgggcactaactgacgctgaggctcgaaagcatgggtagcaaacagg

SEQ ID NO: 98 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgggtggaacaccggtggcgaaggcggccctctgggccgagaccgacgctgaggcgcgaaagctgggggagcgaacagg

SEQ ID NO: 99 - DNA, 253 bp, Akkermansia
tacagaggtctcaagcgttgttcggaatcactgggcgtaaatcgtgcgtaggctgtttcgtcagtcgtgtgtgaaaggcgcgggctcaacccgcggacggcacatgatactgcgagactagagtaatggagggggaaccggaattctcggtgtagcagtgaaatgcgtagagatcgggaggaacacgagtggcgaaggcgggttactggacgttaactgacgctgaggcacgaaggcaaggggagcgaaaggg

SEQ ID NO: 100 - DNA, 253 bp, Akkermansia
tacagaggtctcaagcgttgttcggaatcactgggcgtacagcgtgcgtaggctgtttcgtacgtcgtgtgtgaaaggtgcgggctcaacccgcggactgcacatgatactgcgagactagagtaatggagggggaaccggaattcgcggtgtagcagtgaaaggcgtagatatcgagaggaacacgcgtggcgaaggcgggttcctggacagtaactgacgctgaggcacgaaggccaggggagcgaaaggg

SEQ ID NO: 101 - DNA, 253 bp, Akkermansia
tacagaggtctcaagcgttgttcgggatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcaaaaggg

SEQ ID NO: 102 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacctgatactgcgagactagagtaatggagggggaaccggaattctcggtgtagcggtgaaatgcgtagatattaggaggaacaccagtggcgaaggcggctttctggacgatgactgacgttgaggctcgaaagcgtggggagcaaacagg

SEQ ID NO: 103 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacaccggtggcgaaggcggtcttctgggcctttactgacgctaaagtacgaaagctaggggagcaaacggg

SEQ ID NO: 104 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatgcggaggaacaccgatggcgaaggcaatcccctgggcctgtactgacgctcatgcacgaaagcgtggggagcaaacagg

SEQ ID NO: 105 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggtctctgggccgttactgacgctgaggagcgaaagcgtggggagcgaacagg

SEQ ID NO: 106 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagatatcaagaggaacactcgtggcgaaggcgggttcctggacagtaactgacgctgaggcacgaaagccaggggagcaaaaagg

SEQ ID NO: 107 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggagttctcggtggagcagtgaaatgcgtagatagcgagaggaacactcgtggcgaaggagggttcctggaaagtaacggacgctgaggcgcgaaggccaggggagcgaaaggg

SEQ ID NO: 108 - DNA, 253 bp, Akkermansia
tacgtaggtggcaagcgttgctcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagatatcgagaggaacactcgtggcgaaggcgggttcctggacattaactgacgctgaggcacgaaggtgaggggagcgaaaggg

SEQ ID NO: 109 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattcccggtgtaacggtggaatgtgtagatatcgggaagaacaccaatggcgaaggcaggtctctgggccgttactgacgctgaggagcgaaagcgtggggagcgaacagg

SEQ ID NO: 110 - DNA, 253 bp, Akkermansia
tacgtaggtcccgagcgttgtccggatttattgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 111 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtggagcggtgaaatgagtagatatcgagaggaacagtcgtggcgagggcgggttaatggacattaactgacgctgaggcacgaaggcgaggggagcgaaaggg

SEQ ID NO: 112 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggagccggaattctcggtgtagcggtgaaatgcgtagatatcgagaagaacactgatggcgaaggcaggttcccgggcagtaactgacgctgaggcgcgaaagcgaggggagcgaaaggg

SEQ ID NO: 113 - DNA, 253 bp, Akkermansia
tacagaggtctcaagcgttgttcggaatcactgggcgtaaagcgtgcgtaggctgtttcgtaagtcgtgtgtgaaaggtgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggcgaggggagcaaaaggg

SEQ ID NO: 114 - DNA, 253 bp, Akkermansia
tacgtaggtggcgagcgttatccggaattattgggcgtaaagcgtgcgtaggctgtttcgcacgtcgtgtgtgaaaggcgcgggctcgacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcgcgaaggcgaggggagcgaaaggg

SEQ ID NO: 115 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcagccccctgggataatactgacgctcatgcacgaaagcgtggggagcaaacagg

SEQ ID NO: 116 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagatatcgagaggaacacgcgtggcgaaggcgggtgcctggacattaactgacgcggaggcacgaaggcgaggggagcaaaaggg

SEQ ID NO: 117 - DNA, 253 bp, Akkermansia
tacagaggtctcaagcgttgttcggattcactgggcgtaaagcgtgcgtaggctgtttcgtaagtcgtgtgtgaaaggtgcgggctcaacccgcggacggcacatgatactgcgagactagagtaatggagggggaaccggaattctcggtgtagcggtgaaatgcggagatatcgagaggaacacacgtggcgaaggcaggtacctggacattaactgacgctgaggcacgaaggcgaggggagcgaaaggg

SEQ ID NO: 118 - DNA, 253 bp, Akkermansia
tacagaggtctcaagcgttgttcggaattactgggcgtaaagcgtgcgtaggctgtttcgtaagtcgtgtgtgaaaggcgcgggctcaacctgcggacggcacatgatactgcgagactagagtaatggagggggaaccggaattctcggtgtagcggtgaaatgcgtagagatcgagaggaacaccggtggcgaaggcaggttcctggacattaactgacgctgaggcacgaaggcgaggggagcgaaaggg

SEQ ID NO: 119 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgggaagaacagcgatggcgaaggcagcctgctgggccgcaactgacgctgaggcgcgaaagctaggggagcgagcagg

SEQ ID NO: 120 - DNA, 253 bp, Akkermansia
tacagaggtctcaagcgttgttcggaatcactgggcgtaaagcgtgcgtaggctgtttcgtaagtcgtgtgtgaaaggtgcgggctcaacccgcggacggcacatgatactgcgagactagagtaatggagggggaaccggaattctcggtgtagcggtgaaatgcgtagatatcgagaggaacacccgtggcgaaggcgggttactggactttaactgacgctgaggctcgaaggcgtggggagcgaaaggg

SEQ ID NO: 121 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagatatcgagaggaacactcgtggcgaaggcaggttcctggacattaactgacgctgatgcacgaaggccaggggagcaaaaggg

SEQ ID NO: 122 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagatattaggaggaacaccggtggcgaaggcggctttctggactgacactgacgctgaggcacgaaagcgtggggagcaaacagg

SEQ ID NO: 123 - DNA, 253 bp, Akkermansia
tacgtagggtgcaagcgttaatcggaattactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 124 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatattgggaggaacaccagtagcgaaggcggctttctggactgcaactgacactgaggcacgaaagcgtgggtagcaaacagg

SEQ ID NO: 125 - DNA, 253 bp, Akkermansia
tacagaggtctcaagcgttgttcggaatcactgggcgtaaagcgtgcgcaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggcgaggggagcaaaaggg

SEQ ID NO: 126 - DNA, 253 bp, Akkermansia
tacagaggtctctagcgttgttcggaatcactgggcgtaaagcgtgcgtaggctgtttcgtaagtcgtgtgtgaaaggcgcgggctcaacccgcggacggcacatgatactgcgagactagggtaatggagggagaaccggaattctcggtgtagcagtgaaatgcgtagatagcgagaggaacactcgtggcgaaggcgagttcctggacattaactgacgctgaggcacgaaggccaggggagcaaaaggg

SEQ ID NO: 127 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcagctcaccgggccggaactgacactgatgctcgaaagtgcgggtatcaaacagg

SEQ ID NO: 128 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgcaatgcgtagatagcgagaggaacactcgtggcgaaggcgggtgcctggacattaactgacgctgaggcacgaaggccaggggagcaaaaggg

SEQ ID NO: 129 - DNA, 253 bp, Akkermansia
tacagaggtctcaagcgttgttcggaatcactgggcgtaaagcgtgtgtaggctgtttcgtaagtcgtgtgtgaaaggcgcgggctcaacccgcggacggcacatgatactgcgagactagagtaatggagggggaaccggaattctcggtgtagcagtgaaatgcgtagatattaggaagaacatcagtggcgaaggcggctttctggactgtaactgacactgaggcatgaaagcgtggggagcaaacagg

SEQ ID NO: 130 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcagccatccggcctaatactgacactgaggtgcgaaagcatggggagcaaacagg

SEQ ID NO: 131 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgatgctcgaaagtgtgggtatcaaacagg

SEQ ID NO: 132 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgggaggaacaccagtggcgaaggcgacttactggactgtaactgacgctgaggcgcgaaagtgtggggagcaaacagg

SEQ ID NO: 133 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagcaatggagggggaaccggaattctcggtgtagcagtgaaatgcgtagatatcgagaggaacactcgtggcgaaggcgggttcctggacattaactgacgctgaggcacgaaggcgaggggagcaaaaggg

SEQ ID NO: 134 - DNA, 253 bp, Akkermansia
taaagagggagaaagaggaggtcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 135 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacaccggtggcgaaggcggccacctggacggtaactgacgctgaggtgcgaaagcgtgggtagcaaacagg

SEQ ID NO: 136 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagatatcgagaggaacactcgtggcgaaggcgggttcctgggcagtaactgacgctgaggagcgaaagcatgggtagcgaacagt

SEQ ID NO: 137 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcggtgaaatgcgtagatatcgagaggaacactcgtggcgaaggcggcttcctggacaggaactgacgctgaggcacgaaagccaggggatcaaaaagg

SEQ ID NO: 138 - DNA, 253 bp, Akkermansia
tccctctttctccctcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 139 - DNA, 253 bp, Akkermansia
tacgtagggagcgagcgttgtccggaatcactgggtgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacacgcggggcgaaggcgggttcctggacattaactgacgctgaggcacgaaggccaggggagcgaaaggg

SEQ ID NO: 140 - DNA, 253 bp, Akkermansia
tacgtatgtcgcaagcgttatccggatttattgggcgtaaagcgcgtctaggcggcatagtaagtctgatgtgaaaatgcggggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 141 - DNA, 253 bp, Akkermansia
tacagagttctcaagcgttgttcggaatcactgggcgtaaagcgtgcgtaggctgtttcgtaagtcttgtgtgaaaggcgcgggctcaacccccggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggcgaggggagcaaaaggg

SEQ ID NO: 142 - DNA, 253 bp, Akkermansia
cacgtaagttgcgagcgttgttcggaattattgggcgtaaagggcatgtaggcggttctgcaagtctggtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 143 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtggcagtgaaatgcgtagatatcgagaggaacactcgtggcgaaggcgggttcctggactgcaactgacactgaggcgcgaaagcgtgggtagcaaacagg

SEQ ID NO: 144 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtagtggagggggagccagaattctcggtgtagcggtgaaatgcgtagatatcgagaggaacactagtggcgaaggcaggttcctggacattaactgacgctgaggcgcgaaggcgaggggagcaaaaggg

SEQ ID NO: 145 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggactgcacatgatactgcgagactagagtaatggagggggaaccggaattctcggtgtagcagtgaaatgcgtagatatcgagaggaacactcgtggcgaaggcgggttcctggacattaactgacgctgaggcgcgaaggcgaggggagcaaaaggg

SEQ ID NO: 146 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagagatcgagaggaacactcgtggcgaaggcgggttcctggacattaactgacgctgaggcacgaaggccaggggagcaaaaggg

SEQ ID NO: 147 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatacggaggaacaccagtggcgaaggcggcctgctggacagcaactgacgctgaggcgcgaaagcgtggggagcaaacagg

SEQ ID NO: 148 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcagatgatactgcgagactagagtaatggagggggaaccggaattctcggtgtagcggtgaaatgcgtagatatcgagaggaacactcgtggcgaaggcgggttcctggacattaactgacgctgaggcacgaaggccaggggagcaaaaggg

SEQ ID NO: 149 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaatcggaattccgtgtgtagcggtgaaatgcgtagatatacggaggaacaccagtggcgaaggcggattgctggacagtaactgacgctgaggcgcgaaagcgtggggagcaaacagg

SEQ ID NO: 150 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcggcttgctagccaaagactgacgctcatgcacgaaagcgtggggagcaaatagg

SEQ ID NO: 151 - DNA, 253 bp, Akkermansia
tacagaggtctcaagcgttgttcggaatcactgggcgtaaagcgtgcgtaggctgtttcgtaagtggtgtgagaaagccgcgggctcaacccgcggacggcacatgatactgcgagactagagtaatggagggggaaccggaattctcggtgtagcagtgaaaggcggagatatcgagaggaacactcgtggcgagggcgggttcctggacattaactgacgctgaggcacgaaagcgaggggagcgaaaggg

SEQ ID NO: 152 - DNA, 253 bp, Akkermansia
tacagaggtctcaagcgttgttcggaatcactgggcgtaaagcgtgcgtaggctgtttcgtaagtcgtgtgtgaaagccgcgggctcaacccgcggactgcacatgaaactgcgagactagagtaatggagggggaaccggaattctcggtgtagcagtgaaatgcgtagagatcgagaggaacactcgtggcgaaggcgggttcctggacaataactgacgctgaggcacgaaggccaggggagcgaaaggg

SEQ ID NO: 153 - DNA, 253 bp, Akkermansia
tacgtaggggacaagcgttgtccggaatcactgggcgtaaagcgtgcgtaggctgtttcgtaagtcgtgtgtgaaaggcgcgggctcaacccgcggacggcacatgataccgcgagactagagtaatggagggggaaccggaattctcggtgtagcagtgaaatgcgtagatatcgagaggaacactcgtggcgaaggcgggttcctggacagtaactgacgctgaggcgcgaaggccaggggagcgaaaggg

SEQ ID NO: 154 - DNA, 253 bp, Akkermansia
tacagaggtctcaagcgttgttcggaatcactgggcgtaaagcgtgcgtaggctgtttcgtaagtcgtgtgtgaaagccgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggcgaggggagcaaaaggg

SEQ ID NO: 155 - DNA, 253 bp, Akkermansia
tacagaggtctcaagcgttgttcggaatcactgggcgtaaagcgtgcgtaggctgtttcgtaagtcgtgtgtgaaagtcgcgggctcaacccgcggactgcagatgatactgcgagactagagtaatggagggggaaccggaattctcggtgtagcggtgaaatgcgtagatatcgagaggaacactgatggcgaaggcaggttcctggacattaactgacgctgaggcacgaaggcgaggggagcgaaaggg

SEQ ID NO: 156 - DNA, 253 bp, Akkermansia
tacagaggtctcaagcgttgttcggaatcactgggcgtacagcgtgcgtaggctgtttcgtaagtcgtttgtgaaaggcgcgggctccacccgcggacggcacatgatactgcgagactagagtaatggagggggaaccggaattctcggtgtagcagtgaaatgcgtagatatcgagaggaacactcgtggcgaaggcgggttcatggacattaacggacgttgaggcacgaaggcgaggggagtgaaaggg

SEQ ID NO: 157 - DNA, 253 bp, Akkermansia
tacagaggtctcaagcgttgttcggaatcactgggcgtaaagcgtgcgtaggctgtttcgtacgtcgtgtgtgaaaggcgcgggctcaacccgcggacggcacatgatactgcgagactagagtaatggagggggaaccggaattctcggtgtagcggtgaaatgcgtagatagcgagaggaacactcggggcgaaggcgggttcctggacagtaactgacgctgaggcacgaaagcgaggggagcgaaaagg

SEQ ID NO: 158 - DNA, 253 bp, Akkermansia
tacggagggtgctagcgttaatcggaatcactgggcgtaaagcgcacgtaggctgttatgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 159 - DNA, 253 bp, Akkermansia
tacggaggatccgagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaccactcgtggcgaaggcgggttcctggacattaactgacgctgaggcgcgaaggccaggggagcgaaaggt

SEQ ID NO: 160 - DNA, 253 bp, Akkermansia
tacagaggtctcaagcgttgttcggaatcactgggcgtaaagcgtgcgtaggctgtttcgtaagtcgtgtgtgaaaggtgcgggctcaacccgcggacggcccatgatactgcgagactagagtaatggagggggaaccggaattctcggtgtagcggtgaaatgcgtagatatcgagaggaacactcgtggcgaaggcgggttcctggacattaactgacgctgaggcacgaaagcgaggggagcaaaaggg

SEQ ID NO: 161 - DNA, 253 bp, Akkermansia
taccgaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattagctgacgctgagacacgaaagcgtggggagcaaatagg

SEQ ID NO: 162 - DNA, 253 bp, Akkermansia
tacgtagggggcaagcgttatccggatttactgggtgtaaagggagcgtagacggcgatgcaagtctgaagtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaaggg

SEQ ID NO: 163 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatattaggaggaacaccagtggcgaaggcggctttctggactgtaactgacgttgatgtgcgaaagtgtggggagcaaacagg

SEQ ID NO: 164 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatatggaggaacaccagtggcgaaggcggcttgctagccaaagactgacgctcatgcacgaaagcgtggggagcaaatagg

SEQ ID NO: 165 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattcctagtgtagcggtgaaatgcgtagatattaggaggaacagcagtggcgaaggcggattgctggacggcaactgacggtgaggcgcgaaagtgtggggagcaaacagg

SEQ ID NO: 166 - DNA, 253 bp, Akkermansia
tacggggggtgcgagcgttaatcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggacattaactgacgctgaggcacgaaggcgaggggagcgaaaggg

SEQ ID NO: 167 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgagaggaacactcgtggcgaaggcgggttcctggcatcatactgacactgaggttcgaaagcgtgggtagcaaacagg

SEQ ID NO: 168 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcacgaagaactcctattgcgaaggcagcttgctggagtgtaactgacgctgatgctcgaaagtgtgggtatcaaacagg

SEQ ID NO: 169 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcgggaggaacaccagtggcgaaggcggcttcctggcacacaactgacgctcatgtgcgaaagccagggcagcgaacggg

SEQ ID NO: 170 - DNA, 253 bp, Akkermansia
tacgtaggtggcaagcgttgttcggaatcactgggcgtaaagcgtgcgtaggctgtttcgtaagtcgtgtgtgacaggctcgggctcaacccgcggacggcacatgatactgcgagactagagtaatggagggggaaccggaattctcggtgtagcagtgaaatgcgtagatatcgagaggaacactcgtggcgaaggcgggttactggacagtaactgacgctgaggcacgaaggccaggggagcgaaaggg

SEQ ID NO: 171 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatatggaggaacaccggtggcgaaagcggctctctggtctgtaactgacgctgaggctcgaaagcgtggggagcaaacagg

SEQ ID NO: 172 - DNA, 253 bp, Akkermansia
tacagaggtctcaagcgttgttcggaatcactgggcgtaacgcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatagcgagaggaacactcggggcgaaggcgggttcctggacattaacggacgctgaggcgcgaaggcgaggggatcgaaaggt

SEQ ID NO: 173 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatattaggaagaacaccagtggcgaaggcggctttctggacgacaactgacgctgaggcgcgaaagccaggggagcgaacggg

SEQ ID NO: 174 - DNA, 253 bp, Akkermansia
tacagaggtctcaagcgttgttcggaatcactgggcgtaaagcgtgcgtaggctgtttcgtaagtcgtgtgtgaaaggcgcgggctcaacccgcggacggcacatgatactgcgagactagagtaatggagggggaaccggaattctcggtgtagcagtgaaatgcgtagatatcatgaagaactccgatcgcgaaggcatatgtctggagtgcaactgacgctgaggctcgaaagtgcgggtatcgaacagg

SEQ ID NO: 175 - DNA, 253 bp, Akkermansia muciniphila; identical to SEQ ID NO: 24

SEQ ID NO: 176 - DNA, 19 bp, Artificial sequence
  Single strand DNA oligonucleotide
gtgccagcmgccgcggtaa